(19) United States
(12) Patent Application Publication
(10) Pub. No.: US 2025/0333701 A1
(43) Pub. Date: Oct. 30, 2025

(54) COMPOSITIONS AND METHODS FOR GENERATING HEMATOPOIETIC STEM CELLS (HSCS)

(71) Applicants: Washington University, St. Louis, MO (US); Ospedale San Raffaele S.R.L., Milano (IT); Fondazione Telethon, Rome (IT)

(72) Inventors: Christopher Sturgeon, St. Louis, MO (US); Andrea Ditadi, St. Louis, MO (US)

(21) Appl. No.: 18/968,839

(22) Filed: Dec. 4, 2024

Related U.S. Application Data
(63) Continuation of application No. 17/424,825, filed on Jul. 21, 2021, now abandoned.
(60) Provisional application No. 62/795,550, filed on Jan. 22, 2019.
(60) Provisional application No. 62/903,420, filed on Sep. 20, 2019.

(51) Int. Cl. C12N 5/0789

(52) U.S. Cl. CPC C12N 5/0647; C12N 2500/24; C12N 2500/32; C12N 2500/35; C12N 2500/38; C12N 2501/105; C12N 2501/115; C12N 2501/125; C12N 2501/14; C12N 2501/15; C12N 2501/155; C12N 2501/16; C12N 2501/165; C12N 2501/2306; C12N 2501/2311; C12N 2501/415; C12N 2506/02; C12N 2506/45; C12N 2510/00

(57) ABSTRACT

The present disclosure provides methods for generating hematopoietic progenitor cells. In some embodiments, the methods involve an in vitro or ex vivo cell culture model utilizing rentionic acid signaling for producing hematopoietic progenitor cells from pluripotent stem cells.

### INCORPORATION OF SEQUENCE LISTING

The present application contains a Sequence Listing which has been submitted in .XML format via Patent Center and is hereby incorporated in its entirety. Said Sequence Listing was created on Dec. 4, 2024, XML copy is named 047563_69626000N.xml and is 5.98 kilobytes in size.

### FIELD OF THE TECHNOLOGY

This disclosure generally relates to compositions and methods for producing hematopoietic progenitor cells.

### BACKGROUND

The hematopoietic stem cell (HSC) is pluripotent and ultimately gives rise to all types of terminally differentiated blood cells. The hematopoietic stem cell can self-renew, or it can differentiate into more committed progenitor cells, which progenitor cells are irreversibly determined to be ancestors of only a few types of blood cell. For instance, the hematopoietic stem cell can differentiate into (i) myeloid progenitor cells, which myeloid progenitor cells ultimately give rise to monocytes and macrophages, neutrophils, basophils, eosinophils, erythrocytes, megakaryocytes/platelets, dendritic cells, or (ii) lymphoid progenitor cells, which lymphoid progenitor cells ultimately give rise to T-cells, B-cells, and lymphocyte-like cells called natural killer cells (NK-cells). Once the stem cell differentiates into a myeloid progenitor cell, its progeny cannot give rise to cells of the lymphoid lineage, and, similarly, lymphoid progenitor cells cannot give rise to cells of the myeloid lineage. For a general discussion of hematopoiesis and hematopoietic stem cell differentiation, see Chapter 17, Differentiated Cells and the Maintenance of Tissues, Alberts et al., 1989, Molecular Biology of the Cell, 2nd Ed., Garland Publishing, New York, N.Y.; Chapter 2 of Regenerative Medicine, Department of Health and Human Services, August 2006, and Chapter 5 of Hematopoietic Stem Cells, 2009, Stem Cell Information, Department of Health and Human Services.

In vitro and in vivo assays have been developed to characterize hematopoietic stem cells, for example, the spleen colony forming (CFU-S) assay and reconstitution assays in immune-deficient mice. Further, presence or absence of cell surface protein markers defined by monoclonal antibody recognition have been used to recognize and isolate hematopoietic stem cells. Such markers include, but are not limited to, Lin, CD34, CD38, CD43, CD45RO, CD45RA, CD59, CD90, CD109, CD117, CD133, CD166, and HLA DR, and combinations thereof. See Chapter 2 of Regenerative Medicine, Department of Health and Human Services, August 2006, and the references cited therein.

Hematopoietic stem cells have therapeutic potential as a result of their capacity to restore blood and immune cells in transplant recipients. Specifically, autologous allogeneic transplantation of HSC can be used for the treatment of patients with inherited immunodeficient and autoimmune diseases and diverse hematopoietic disorders to reconstitute the hematopoietic cell lineages and immune system defense. Human bone marrow transplantation methods are currently used as therapies to treat various diseases like: cancers, leukemia, lymphoma, cardiac failure, neural disorders, auto-immune diseases, immunodeficiency, metabolic or genetic disorders. Several challenges remain to be addressed prior to developing and applying large scale cell therapies, for example, for these procedures, a large number of stem cells must be isolated to ensure that there are enough HSCs for engraftment. The number of HSCs available for treatment is a clinical limitation.

### BRIEF DESCRIPTION OF THE FIGURES

The application file contains at least one drawing executed in color. Copies of this patent application publication with color drawing(s) will be provided by the Office upon request and payment of the necessary fee.

FIG. 1A-1L show scRNA-seq reveals unexpected heterogeneity in hPSC-derived definitive hemogenic mesoderm FIG. 1A shows a UMAP plot of transcriptionally distinct clusters within WNTi or WNTd day 3 of differentiation cultures, obtained. FIG. 1B shows the expression of KDR, GYPA, and CDX4 within differentiation cultures. FIG. 1C shows UMAP visualizing distinct clusters within WNTd differentiation cultures, projection of germ layer type onto each cluster, and dot plot visualizing expression of germ layer-specific genes within each identified cluster. FIG. 1D shows UMAP visualizing CDX4+(green) and CDX4^{neg} (blue) mesodermal cluster. FIG. 1E shows a UMAP for ALDH1A2 and CYP26A1. FIG. 1F shows a UMAP visualizing CXCR4 expression. FIG. 1G shows pseudotime single cell trajectory of WNTd differentiation cultures, predicted temporal progression from early (purple) to late (yellow) differentiation events and predicted germ layer identity. FIG. 1H shows a violin plot visualizing the expression of CXCR4 and CDX4 within branches 6 and 7. FIG. 1I shows a heatmap of scRNA-seq dataset showing expression of CDX4, CXCR4, ALDH1A2, and CYP26A1 over pseudotime and following the branching of mesoderm into two distinct populations. FIG. 1J shows CXCR4 is expressed within hPSC-derived mesoderm in a WNT-dependent manner, representative flow cytometric analysis of KDR and CXCR4 expression on day 3 of differentiation, following WNTi or WNTd differentiation conditions, and average percentage of CXCR4+ cells within each day 3 culture within both H1 (light blue) and hPSC-1 (dark blue) mesoderm. FIG. 1K shows representative Aldefluor (ALDF) flow cytometric analysis within KDR+ cells, with DEAB (pan-ALDH inhibitor) serving as a negative control. FIG. 1L shows shows representative flow cytometric analysis for endothelial markers CD34, CD144 (VE-Cadherin), and TEK (TIE2) within KDR+ cells (unstained in inset). n≥3, SEM, t-test, ***p<0.001, ****p<0.0001.

FIG. 2A-2D show that CXCR4^{neg} and CXCR4+ mesoderm gives rise to hemogenic endothelium in a RA-independent and RA-dependent manner, respectively. FIG. 2A shows separation of mesodermal progenitors of hemogenic endothelium, based on CXCR4 cell surface expression, representative FACS gating scheme of KDR+ mesoderm for presence or absence CXCR4 expression, within WNTd day 3 of differentiation cultures, representative FACS gating scheme of CD34 and CD43 expression, following 5 days of culture after KDR+ mesoderm isolation, and representative flow cytometric analyses of T-lymphoid potential of CD34+CD43^{neg} populations, T cell potential is positively identified by the presence of a CD4+CD8+ population following 21+ days of OP9-DL4 coculture, while an absence of potential is identified by an absence of CD45+ lymphocytes. FIG. 2B shows quantification of the definitive erythro-myeloid CFC potential from different hemogenic endothelial populations; n=3. FIG. 2C shows the specification of RA-dependent hemogenic endothelium is stage-specific. Quantification of definitive erythro-myeloid CFC potential of CD34+CD43^{neg} populations, following ROH treatment on either day 3, 4, or 5. n=3. FIG. 2D shows the quantification of definitive erythro-myeloid CFC potential of CD34+CD43^{neg} cells, following ATRA treatment on day 3 of differentiation, as in (A). Isolated day 3 of differentiation (i) WNTd CXCR4+, (ii) WNTd CXCR4neg, or (iii) WNTi CD235a+ mesoderm, were treated with various concentrations of ATRA immediately following isolation, cultured further as in (A), and resultant CD34+ cells were isolated and assessed for hematopoietic potential, as in (B). n≥4, SEM, ANOVA, **p<0.01, ***p<0.001, ****p<0.0001.

FIG. 3A-3C show HE with different ontogenic origins can be specified from hPSCs. FIG. 3A shows a heatmap visualizing the relative expression of HOXA genes within WNTi HE, RAi HE, RAd HE, and fetal endothelium. FIG. 3B shows a heatmap visualizing the similarity between scRNA-seq and bulk RNA-seq, comparing each arterial endothelial cell (AEC) and HE cell (HEC) from Carnegie Stage (CS)10, CS11, and CS13 human embryos18 to CXCR4ⁿe9 and CXCR4+ mesoderm and WNTi, RAi, and RAd HE RNA-seq datasets using SingleR. Similarity scores are relative Spearman coefficients. FIG. 3C shows average similarity scores for each fetal HE or endothelial population compared to each hPSC-derived population, as indicated.

FIG. 4 shows different mesodermal populations can be obtained from hPSCs, based on stage-specific manipulation of ACTIVIN and WNT signaling. Representative flow cytometric analysis of KDR, CD235a, and CXCR4 expression on day 3 of differentiation, following CHIR99021 and SB431542 treatment (top) or IWP2 and ACTIVIN A treatment (bottom).

FIG. 5 shows definitive erythro-myeloid hematopoietic potential of bulk differentiation cultures, treated with either DEAB or ROH on day 3 of differentiation. n=1.

FIG. 6 shows a revised roadmap of hematopoietic development from hPSCs. hPSCs (day 0) are driven towards primitive streak (day 2) using BMP4. CD235a+ CYP26A1+ mesoderm that give rise to HOXA^{low/neg} extra-embryonic-like HE is patterned in a WNT-independent (WNTi), ACTIVIN/NODAL-dependent manner. Nascent mesoderm patterned in a WNT-dependent (WNTd) manner contains two distinct progenitors to HOXA+ intra-embryonic-like HE. CXCR4^{neg} CYP26A1+ mesoderm gives rise to RAi HE, while CXCR4+ALDH1A2+ mesoderm gives rise to RAd HE. All 3 ontogenically-distinct HE populations undergo the EHT in a NOTCH-dependent manner but with functionally-distinct hematopoietic progeny.

FIG. 7A-7B show proposed models of hematopoietic development. FIG. 7A maturational model, wherein all hematopoiesis originates from a common mesodermal progenitor. FIG. 7B distinct origin model, with each wave originating from unique mesodermal subsets.

FIG. 8A-8C show hPSC-derived WNT-dependent HE is multipotent but has low medial HOXA expression. FIG. 8A shows clonal multi-lineage assay of hPSC-derived HE. Single cells are isolated by FACS into 96 well plates with OP9-DL4 stroma. HE is cultured for 7 days to allow for the EHT to occur, followed by half the well plated in methylcellulose, the other half onto fresh stroma under T-lymphoid promoting conditions. Clones can be scored for uni-, bi-, or multi-lineage capacity. FIG. 8B shows differences in HOXA gene expression between in vitro and in vivo CD34+ cells. hPSC-derived HE and independently generated hPSC-derived HE, was compared by RNA-seq against 5th week fetal human AGM endothelium (containing HE and committed endothelium). n=3. Mean±SEM. ***p>0.001. HOXA11-13 had AGM RPMKs of “0” and were excluded from analysis. FIG. 8C shows qRT-PCR analysis of HOXA genes within CXCR4+-derived CD34+ cells following ROH treatment on day 3 of differentiation, in comparison to CXCR4^{neg}-derived CD34+ cells. Mean±SEM. n=4. * p<0.05. ** p<0.001.

FIG. 9 shows RA-dependent HE gives rise to progenitors that persist in a xenograft. Representative flow cytometric analysis of the peripheral blood from 2 different recipients, 8 weeks post-intrahepatic injection.

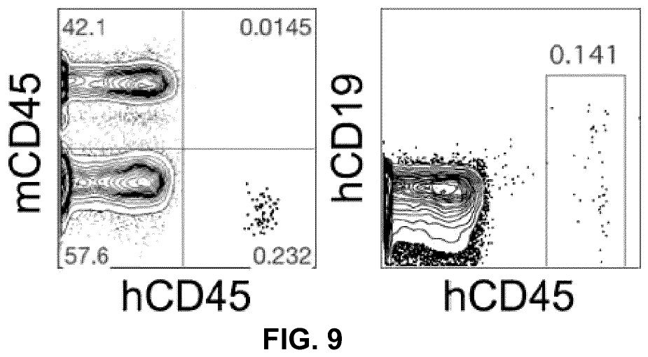

### DETAILED DESCRIPTION

The generation of the hematopoietic stem cells (HSCs) from human pluripotent stem cells (hPSCs) is a major goal for regenerative medicine. HSCs derive from hemogenic endothelium (HE) in a NOTCH and retinoic acid (RA)-dependent manner. While a WNT-dependent (WNTd) patterning of nascent hPSC mesoderm specifies clonally multipotent NOTCH-dependent definitive HE and this HE is functionally unresponsive to RA. The present disclosure establishes that WNTd mesoderm, prior to HE specification, is actually comprised of two distinct KDR+ CD34^{neg} populations. CXCR4^{neg} CDX4+ mesoderm gives rise to HOXA+ multilineage definitive HE, in an RA-independent manner, while CXCR4+ALDH1A2+ mesoderm gives rise to multilineage definitive hemogenic endothelium in a stage-specific, RA-dependent manner. Further, this RA-dependent HE is transcriptionally similar to primary fetal HOXA+ endothelium. This revised model of human hematopoietic development provides new resolution to the mesodermal origins of the multiple waves of hematopoiesis.

The present disclosure is based, at least in part, on the discovery of an in vitro platform to produce definitive hemogenic endothelium. In particular, the present disclosure provides retinoic acid (RA)-dependent definitive hematopoietic progenitors. As described herein, the in vitro generation of definitive hematopoietic progenitors can provide either patient-specific cell-based therapeutics, or, “off-the-shelf” universal donor products. The disclosed methodology to produce in vitro derived HSCs can be easily implemented, is robust, and can be used in the development of various clinical and industrial applications, such as but not limited to: cell-based therapies for a variety of hematological conditions; scalable generation of lymphoid progenitors and terminally differentiated lymphocytes for adoptive immunotherapy; scalable generation of megakaryocyte progenitors and/or platelets for transfusion; scalable generation of erythroid progenitors and/or mature erythrocytes for transfusion; the generation of HSCs as a substitute for bone marrow transplantation; drug/toxicity screening on any progenitor or terminally differentiated hematopoietic cell; gene therapy; or gene-correction and allogeneic transplant of patient-derived hPSCs. These insights provide the basis for accurate disease modeling studies and the de novo specification of HSCs.

Additional aspects of the disclosure are described below.

### (I) Methods of Producing Hematopoietic Progenitors

Aspects described herein stem from, at least in part, development of methods that efficiently direct differentiation of pluripotent stem (PS) cells into hematopoietic progenitors. In particular, the present disclosure provides, inter alia, an in vitro or ex vivo culturing process for producing a population of definitive hemogenic endothelium in a stage-specific, RA-dependent manner. Further, this RA-dependent HE is transcriptionally and functionally similar to primary fetal endothelium, including harboring multi-lineage potential. In some embodiments, this culturing process may involve multiple differentiation stages (e.g., 2, 3, or more). Alternatively, or in addition, the culturing process may involve culture of the cells in the presence of a compound which activates retinoic acid signaling. In some embodiment, the total time period for the in vitro or ex vivo culturing process described herein can range from about 6-14 days (e.g., 7-13 days, 7-12 days, or 8-11 days). In one example, the total time period is about 8 days.

In some embodiments, the methods for producing hematopoietic progenitors as disclosed herein may include multiple differentiation stages (e.g., 2, 3, 4, or more). For example, a mesoderm differentiation step, e.g., the culturing of the pluripotent stem cells under differentiation conditions to obtain cells of the mesoderm, a hematopoietic specification step, e.g., the culturing of the obtained mesoderm cells under differentiation conditions to obtain the hematopoietic progenitor cells. In some aspects, the present disclosure includes additional differentiation stages, for example a erythroid maturation step, a myeloid maturation step and/or a lymphoid maturation step.

Existing methods for producing human hematopoietic cells often result in functionally distinct HE populations, which have contributed to difficulties in understanding the physiological relevance of human pluripotent stem cell (hPS) cells-derived hematopoiesis. This is because, as until recently, hPS cells differentiation methods could not discriminate between the progenitors of these various programs. The generation of definitive hematopoietic progenitors from human pluripotent stem cells (hPSCs) is a goal for both regenerative medicine and private industry scientists. However, to ensure that these hematopoietic progenitors faithfully recapitulate the functional behavior(s) of those found in pre-/post-natal and adult humans, the presently disclosed hPSC-derived progenitors have been derived from the developmental programs which occur during embryogenesis. The in vitro or ex vivo model described herein can provide a reliable source of hematopoietic progenitor cells. The pluripotent stem (PS) cell-derived hematopoietic progenitors can be used in various applications, including, e.g., but not limited to, as an in vitro model for hematopoiesis, related diseases or disorders, drug discovery and/or developments.

Accordingly, embodiments of various aspects described herein relate to methods for generation of hematopoietic progenitors from PS cells, cells produced by the same, and methods of use.

### (a) Pluripotent Stem Cells

In some embodiments, the in vitro or ex vivo culturing system disclosed herein may use pluripotent stem cells (e.g., human pluripotent stem cells) as the starting material for producing hematopoietic progenitor cells. As used herein, “pluripotent” or “pluripotency” refers to the potential to form all types of specialized cells of the three germ layers (endoderm, mesoderm, and ectoderm); and is to be distinguished from “totipotent” or “totipotency”, that is the ability to form a complete embryo capable of giving rise to offsprings. As used herein, “human pluripotent stem cells” (hPS) cells refers to human cells that have the capacity, under appropriate conditions, to self-renew as well as the ability to form any type of specialized cells of the three germ layers (endoderm, mesoderm, and ectoderm). hPS cells may have the ability to form a teratoma in 8-12 week old SCID mice and/or the ability to form identifiable cells of all three germ layers in tissue culture. Included in the definition of human pluripotent stem cells are embryonic cells of various types including human embryonic stem (hES) cells, (see, e.g., Thomson et al. (1998), Heins et. al. (2004), as well as induced pluripotent stem cells [see, e.g. Takahashi et al., (2007); Zhou et al. (2009); Yu and Thomson in Essentials of Stem Cell Biology (2nd Edition]. The various methods described herein may utilize hPS cells from a variety of sources. For example, hPS cells suitable for use may have been obtained from developing embryos by use of a nondestructive technique such as by employing the single blastomere removal technique described in e.g. Chung et al (2008), further described by Mercader et al. in Essential Stem Cell Methods (First Edition, 2009). Additionally or alternatively, suitable hPS cells may be obtained from established cell lines or may be adult stem cells.

In some aspects, the pluripotent stem cells for use according to the disclosure may be human embryonic stem cells. Various techniques for obtaining hES cells are known to those skilled in the art. In some instances, the hES cells for use according to the present disclosure are ones, which have been derived (or obtained) without destruction of the human embryo, such as by employing the single blastomere removal technique known in the art. See, e.g., Chung et al., Cell Stem Cell, 2(2):113-117 (2008), Mercader et al., Essential Stem Cell Methods (First Edition, 2009). Suitable hES cell lines can also be used in the methods disclosed herein. Examples include, but are not limited to, cell lines H1, H9, SA167, SA181, SA461 (Cellartis A B, Goteborg, Sweden) which are listed in the NIH stem cell registry, the UK Stem Cell bank and the European hESC registry and are available on request. Other suitable cell lines for use include those established by Klimanskaya et al., Nature 444:481-485 (2006), such as cell lines MA01 and MA09, and Chung et al., Cell Stem Cell, 2(2):113-117 (2008), such as cell lines MA126, MA127, MA128 and MA129, which all are listed with the International Stem Cell Registry (assigned to Advanced Cell Technology, Inc. Worcester, MA, USA).

Alternatively, the pluripotent stem cells for use in the methods disclosed herein may be induced pluripotent stem cells (iPS) cells such as human iPS cells. As used herein “hiPS cells” refers to human induced pluripotent stem cells. hiPS cells are a type of pluripotent stem cells derived from non-pluripotent cells—typically adult somatic cells—by induction of the expression of genes associated with pluripotency, such as SSEA-3, SSEA-4, TRA-1-60, TRA-1-81, Oct-4, Sox2, Nanog and Lin28. Various techniques for obtaining such iPS cells have been established and all can be used in the present disclosure. See, e.g., Takahashi et al., Cell 131(5):861-872 (2007); Zhou et al., Cell Stem Cell. 4(5):381-384 (2009); Yu and Thomson in Essentials of Stem Cell Biology (2nd Edition, Chapter 4)]. It is also envisaged that the hematopoietic progenitor cells may also be derived from other pluripotent stem cells such as adult stem cells, cancer stem cells or from other embryonic, fetal, juvenile or adult sources.

In an exemplary embodiment, human pluripotent stem cells, (wherein hPS cells can comprise both human embryonic stem cells (hES) cells and human induced pluripotent stem cells (hiPS) cells) can be cultured until about 70% confluence. These cells can be removed from these conditions, dissociated into clumps (termed “embryoid bodies”), and then further cultured under hypoxic conditions (about 5% O₂, 5% CO₂) in defined serum-free differentiation media.

In some embodiments, ES cell culture may be grown on one layer of feeder cells. “Feeder cells” refer to a type of cell, which can be second species, when being co-cultured with another type of cell. Feeder cells are generally derived from embryo tissue or tire tissue fibroblast. Embryo is collected from the CF1 mouse of pregnancy 13 days, is transferred in 2 ml trypsase/EDTA, then careful chopping, 37 DEG C incubate 5 minutes. 10% FBS is added, so that fragment is precipitated, cell increases in 90% DMEM, 10% FBS and 2 mM glutamine. The feeder cells offer a growing environment for the ES cells. Certain form of ES cells can use, for example, primary mouse embryonic fibroblast or infinite multiplication mouse embryonic fibroblasts. In order to prepare feeder layer, irradiated cells may be used to support the ES cells (about 3000 rad γ-radiation will inhibit proliferation).

In some embodiments, the PS cells are removed from the feeder cells and cultured in serum free defined media for about 24 hours to generate embryoid bodies. Term “embryoid” is synonymous with “aggregation”, refers to differentiated and neoblast aggregation, which appears in ES cells. It is maintained in undue growth or the culture that suspends in monolayer cultures. Embryoid is different cell types (generally originating from different germinal layers) Mixture, can according to morphological criteria distinguish and available immunocytochemistry detect cell marking. In some embodiments, the PS cells are cultured in a cell culture vessel coated with at least one extracellular matrix protein (e.g., laminin or Matrigel) to generate embryoid bodies.

### (b) Differentiation of Pluripotent Stem Cells

The in vitro or ex vivo culturing system disclosed herein may involve a step of differentiation to differentiate any of the PS cells disclosed herein to hematopoietic progenitor cells.

Suitable conditions for mesoderm differentiation are known in the art (e.g., Sturgeon et al., Nat Biotechnol.; 32(6):554-61 (2014)) and/or disclosed in Examples below. As used herein “mesoderm” and “mesoderm cells (ME cells)” refers to cells exhibiting protein and/or gene expression as well as morphology typical to cells of the mesoderm or a composition comprising a significant number of cells resembling the cells of the mesoderm. The mesoderm is one of the three germinal layers that appears in the third week of embryonic development. It is formed through a process called gastrulation. There are three important components, the paraxial mesoderm, the intermediate mesoderm and the lateral plate mesoderm. The paraxial mesoderm forms the somitomeres, which give rise to mesenchyme of the head and organize into somites in occipital and caudal segments, and give rise to sclerotomes (cartilage and bone), and dermatomes (subcutaneous tissue of the skin). Signals for somite differentiation are derived from surroundings structures, including the notochord, neural tube and epidermis. The intermediate mesoderm connects the paraxial mesoderm with the lateral plate, eventually it differentiates into urogenital structures consisting of the kidneys, gonads, their associated ducts, and the adrenal glands. The lateral plate mesoderm give rise to the heart, blood vessels and blood cells of the circulatory system as well as to the mesodermal components of the limbs.

Some of the mesoderm derivatives include the muscle (smooth, cardiac and skeletal), the muscles of the tongue (occipital somites), the pharyngeal arches muscle (muscles of mastication, muscles of facial expressions), connective tissue, dermis and subcutaneous layer of the skin, bone and cartilage, dura mater, endothelium of blood vessels, red blood cells, white blood cells, and microglia, the kidneys and the adrenal cortex.

ME cells may generally be characterized, and thus identified, by a positive gene and protein expression of the markers KDR/VEGFR2, and lack of expression of CD235a. Within this KDR+ CD235a^{neg} population, two mesodermal subsets can be identified by the expression of CXCR4/CD184. The emergence of this CXCR4+ population can be enhanced by the application of stage-specific WNT signal activation from about days 2 to 4 of differentiation or about days 2 to 3, as described below. Gene expression analyses have identified that the CXCR4^{neg} population expresses the gene CYP26A1, which suggests that it will not be responsive to retinoic acid signaling (RA). In contrast, it was discovered that the CXCR4+ population expresses the gene ALDH1A2, suggesting it will convert retinol into RA, and subsequently engage RA-dependent cellular differentiation. This enzyme is expressed and is active, as evidenced by Aldefluor uptake and conversion to a fluorescent compound.

Generally, in order to obtain ME cells, PS cells such as hPS cells can be cultured in a differentiation medium comprising L-glutamine, ascorbic acid, monothioglycerol, and a differentiation inducer such as transferrin. The differentiation medium may be optionally further supplemented with one or more growth factors, such as a fibroblast growth factor (FGF) (e.g., FGF1, FGF2 and FGF4), and one or more bone morphogenic proteins (BMP), such as BMP2 and BMP4. As used herein, the term “FGF” means fibroblast growth factor, preferably of human and/or recombinant origin, and subtypes belonging thereto are e.g. “bFGF” (means basic fibroblast growth factor, sometimes also referred to as FGF2) and FGF4. “aFGF” means acidic fibroblast growth factor (sometimes also referred to as FGF1). As used herein, the term “BMP” means Bone Morphogenic Protein, preferably of human and/or recombinant origin, and subtypes belonging thereto are e.g. BMP4 and BMP2. The concentration of the one or more growth factors may vary depending on the particular compound used. The concentration of FGF2, for example, is usually in the range of about 2 to about 50 ng/ml, such as about 2 to about 20 ng/ml. FGF2 may, for example, be present in the specification medium at a concentration of 9 or 10 ng/ml. The concentration of FGF1, for example, is usually in the range of about 50 to about 200 ng/ml, such as about 80 to about 120 ng/ml. FGF1 may, for example, be present in the specification medium at a concentration of about 100 ng/ml. The concentration of FGF4, for example, is usually in the range of about 20 to about 40 ng/ml. FGF4 may, for example, be present in the specification medium at a concentration of about 30 ng/ml. The concentration of the one or more BMPs, is usually in the range of about 50 to about 300 ng/ml, such as about 50 to about 250 ng/ml, about 100 to about 250 ng/ml, about 150 to about 250 ng/ml, about 50 to about 200 ng/ml, about 100 to about 200 ng/ml or about 150 to about 200 ng/ml. The concentration of BMP2, for example, is usually in the range of about 2 to about 50 ng/ml, such as about 10 to about 30 ng/ml. BMP2 may, for example, be present in the hepatic specification medium at a concentration of about 20 ng/ml.

In one aspect, from about days 0-3 of differentiation, embryoid bodies can be exposed to recombinant human BMP4. On about days 1-3 of differentiation, bFGF can be added to the differentiation media.

In some embodiments, the differentiation media comprises an activin, such as activin A or B. The concentration of activin is usually in the range of about 50 to about 200 ng/ml, such as about 80 to about 120 ng/ml. Activin may, for example, be present in the differentiation medium at a concentration of about 90 ng/ml or about 100 ng/ml. As used herein, the term “Activin” is intended to mean a TGF-beta family member that exhibits a wide range of biological activities including regulation of cellular proliferation and differentiation such as “Activin A” or “Activin B”. Activin belongs to the common TGF-beta superfamiliy of ligands. The differentiation medium may further comprise an inhibitor of the activin receptor-like kinase receptors, ALK5, ALK4 and ALK7, such as SB431542. The concentration of the ALK5, ALK4 and ALK7 inhibitor is usually in the concentration of about 1 μM to about 12 μM, such as about 3 μM to about 9 μM. The differentiation media may comprise a GSKβ-inhibitor, such as, e.g., CHIR99021 or CHIR98014, or an activator of WNT signaling, such as WNT3A.

The concentration of the activator of WNT signaling is usually in the range of about 0.05 to about 90 ng/ml, such as about 50 ng/ml. As used herein, “activator of WNT signaling” refers to a compound which activates WNT signaling. The concentration of the GSKp inhibitor, if present, is usually in the range of about 0.1 to about 10 μM, such as about 0.05 to about 5 μM.

The concentration of serum, if present, is usually in the range of about 0.1 to about 2% v/v, such as about 0.1 to about 0.5%, about 0.2 to about 1.5% v/v, about 0.2 to about 1% v/v, about 0.5 to 1% v/v or about 0.5 to about 1.5% v/v. Serum may, for example, if present, in the differentiation medium may be at a concentration of about 0.2% v/v, about 0.5% v/v or about 1% v/v. In one aspect, the differentiation medium omits serum and instead comprises a suitable serum replacement.

The culture medium forming the basis for the differentiation medium may be any culture medium suitable for culturing PS cells and is not particularly limited. For example, base media such as StemPro-34 media, RPMI 1640 or advanced medium, Dulbecco's Modified Eagle Medium (DMEM), Iscove's Modified Dulbecco's Media (IMDM) F-12 Medium (also known as Ham's F-12), or MEM may be used. Thus, the differentiation medium may be StemPro-34 media or advanced medium comprising or supplemented with the above-mentioned components. In some embodiments, the base media may be a blend of two or more suitable culture medias, for example, the base media may be a blend of IMDM and F-12. In some embodiments, the differentiation medium may be DMEM or a blend comprising DMEM comprising or supplemented with the above-mentioned components. The differentiation medium may thus also be MEM medium or a blend comprising MEM comprising or supplemented with the above-mentioned components. In some embodiments, the differentiation medium may be IMDM or a blend comprising IMDM comprising or supplemented with the above-mentioned components. In some embodiments, the differentiation medium may be F-12 or a blend comprising F-12 comprising or supplemented with the above-mentioned components.

In some embodiments, the differentiation medium comprises, consists essentially of, or consists of, a base medium supplemented with L-glutamine, ascorbic acid, monothioglycerol, transferrin and BMP-4. In other embodiments, the differentiation medium comprises, consists essentially of, or consists of, a base medium supplemented with L-glutamine, ascorbic acid, monothioglycerol, transferrin, BMP-4 and bFGF. In still other embodiments, the differentiation medium comprises, consists essentially of, or consists of, a base medium supplemented with L-glutamine, ascorbic acid, monothioglycerol, transferrin, BMP-4, bFGF, an ALK5, ALK4 and ALK7 inhibitor, and a GSKβ-inhibitor. In another embodiment, the differentiation medium comprises, consists essentially of, or consists of a base medium, 2 mM L-glutamine, 1 mM ascorbic acid, monothioglycerol, 150 μg/mL transferrin and BMP-4. In yet another embodiment, the differentiation medium comprises, consists essentially of, or consists of, a base medium, 2 mM L-glutamine, 1 mM ascorbic acid, monothioglycerol, 150 μg/mL transferrin, BMP-4 and 5 ng/mL bFGF. In still yet another embodiment, the differentiation medium comprises, consists essentially of, or consists of, a base medium, 2 mM L-glutamine, 1 mM ascorbic acid, monothioglycerol, 150 μg/mL transferrin, BMP-4 and 5 ng/mL bFGF, 6 μM SB431542, and 3 μM CHIR99021.

The PS cells are normally cultured for up to 3-4 days in suitable differentiation medium in order to obtain mesoderm cells. For example, from about days 0-3 of differentiation, embryoid bodies can be exposed to recombinant human BMP4. On about days 1-3 of differentiation, bFGF can be added to the differentiation media. On day 2, fresh media can be replaced, with the addition of a WNT signaling stimulating agent (a GSK3b antagonist or inhibitor, such as CHIR99021 or analogs thereof, such as CHIR98014; a recombinant WNT protein; or a WNT agonist) and ACTIVIN/NODAL signaling suppressing agent (e.g., an ALK inhibitor, such as SB-431542 or a small molecule TGFb inhibitor). In some embodiments, the PS cells are cultured in a cell culture vessel coated with at least one extracellular matrix protein (e.g., laminin or Matrigel) during contact with the differentiation medium. The PS cells may be dissociated and collected in suspension (e.g., through contact with TrypLE), if needed.

### (c) Hematopoietic Progenitor Specification

Following the mesoderm differentiation step, the obtained mesoderm cells can be further cultured in a hematopoietic progenitor specification medium to obtain hematopoietic progenitor cells. As used herein, “hematopoietic progenitors” or “hematopoietic stem cells” mean definitive hematopoietic stem cells that are capable of engrafting a recipient of any age post-birth. As described above, hematopoietic progenitors can be derived from: an embryo (e.g., aorta-gonad-mesonephros region of an embryo), embryonic stem cells (ESC), induced pluripotent stem cells (iPSC), or reprogrammed cells of other types (non-pluripotent cells of any type reprogrammed into HSCs). The hematopoietic progenitor cells of the disclosure are not fetal liver HSC, adult peripheral blood HSC or umbilical cord blood HSC. “Hematopoietic progenitors” may generally be characterized, and thus identified, by one or more of a gene or protein expression of CD34+CD43^{neg} CD73^{neg} CD184^{neg}. The hematopoietic progenitor cells can be a hemogenic endothelial (HE) population that is capable of multi-lineage definitive hematopoiesis, at a clonal level.

In general, in order to obtain hematopoietic progenitor cells, mesoderm cells, for example, mesoderm cells as described above, are further cultured in a hematopoietic differentiation medium comprising one or more growth factors, such as a fibroblast growth factor (FGF) (e.g., FGF1, FGF2 and FGF4), one or more vascular endothelial growth factor (VEGF), and a retinoic acid signaling agent. In some embodiments, the retinoic acid can be retinol (ROH), a retinoic acid, such as all-trans-retinoic acid (ATRA), a retinoic acid receptor (RAR) agonist, a RAR alpha (RARA) agonist (e.g., AM580), a RAR beta (RARB) agonist (e.g., BMS453), or a RAR gamma (RARG) agonist (e.g., CD1530). As another example, the RA signaling agent signals for the specification of definitive HE. The concentration of the one or more growth factors may vary depending on the particular compound used. The concentration of bFGF, for example, is usually in the range of about 1 to about 10 ng/ml, such as about 2 to about 8 ng/ml. bFGF may, for example, be present in the specification medium at a concentration of 3 or 7 ng/ml. The concentration of VEGF, for example, is usually in the range of about 2 to about 50 ng/ml, such as about 2 to about 20 ng/ml. VEGF may, for example, be present in the specification medium at a concentration of 9 or 15 ng/ml. The concentration of the one or more RA signaling agent, is dependent on the RA signaling agent used, usually in the range of about 1 to about 10 μM, such as about 2 to about 8 μM, about 3 to about 7 μM. The specification medium may include other factors such as stem cell factor (SCF), Interleukin-6, 3, and 11, insulin growth factors such as IGF-1, and erythropoietin (EPO). SCF, when present, is included at a concentration between about 1 to about 10 ng/ml, such as about 2 to about 8 ng/ml. SCF may, for example, be present in the specification medium at a concentration of 3 or 7 ng/ml. Interleukin when present, when present, is included at a concentration between about 1 ng/mL to about 20 ng/mL, such as about 5 ng/ml to about 10 ng/ml. EPO, when present, is included at a concentration between about 1 U/mL to about 3 U/mL.

In some embodiments, the specification medium comprises, consists essentially of, or consists of, a base medium supplemented with a fibroblast growth factor, a vascular endothelial growth factor (VEGF), and a retinoic acid signaling agent. In another embodiment, the specification medium comprises, consists essentially of, or consists of a base medium, 5 ng/mL bFGF, 15 ng/mL VEGF, and 5 μM retinol. In another aspect, the specification medium consists essentially of, or consists of, a base medium supplemented with IL-6, IGF-1, SCF, EPO, and retinol. In another aspect, the specification medium consists essentially of, or consists of, a base medium supplemented with 10 ng/mL IL-6, 25 ng/ml IGF-1, 5 ng/mL SCF, 2 U/mL EPO, and 5 ng/mL retinol.

The culture medium forming the basis for the hematopoietic specification medium may be any culture medium suitable for culturing mesodermal cells and is not particularly limited. For example, the culture medium forming the basis for the specification medium may be any culture medium suitable for culturing ME cells and is not particularly limited. For example, base media such as StemPro-34 media, RPMI 1640 or advanced medium, Dulbecco's Modified Eagle Medium (DMEM), Iscove's Modified Dulbecco's Media (IMDM) F-12 Medium (also known as Ham's F-12), or MEM may be used. Thus, the differentiation medium may be StemPro-34 media or advanced medium comprising or supplemented with the above-mentioned components. In some embodiments, the base media may be a blend of two or more suitable culture medias, for example, the base media may be a blend of IMDM and F-12. In some embodiments, the differentiation medium may be DMEM or a blend comprising DMEM comprising or supplemented with the above-mentioned components. The differentiation medium may thus also be MEM medium or a blend comprising MEM comprising or supplemented with the above-mentioned components. In some embodiments, the differentiation medium may be IMDM or a blend comprising IMDM comprising or supplemented with the above-mentioned components. In some embodiments, the differentiation medium may be F-12 or a blend comprising F-12 comprising or supplemented with the above-mentioned components. In some embodiments, the ME cells are cultured in a cell culture vessel coated with at least one extracellular matrix protein (e.g., laminin) during contact with the hepatic specification medium.

For specification into hematopoietic progenitor cells, ME cells are normally cultured for up to 3 days in specification medium comprising bFGF, VEGF, and retinoic acid signaling agent. The ME cells may then, for example, be cultured in a specification medium comprising IL-6, IGF-1, IL-11, SCF, EPO, and a retinoic acid signaling agent for an additional 2 days to about 5 days. In some embodiments, the ME cells are maintained in the cell culture vessel optionally coated with at least one extracellular matrix protein, during specification to hematopoietic progenitor cells.

When isolated by fluorescence-activated cell sorting (FACS), the mesoderm KDR+ CXCR4^{neg} cell population, can similarly give rise to a CD34+CD43^{neg} HE population. This CD34+CD43^{neg} HE population is capable of multi-lineage definitive hematopoiesis. The addition of a RA inhibitor at any stage of this differentiation process, such as DEAB, was discovered to have no negative impact resultant definitive hematopoietic specification. Therefore, the definitive hematopoietic progenitors are derived from a KDR+ CXCR4^{neg} mesodermal population, which expresses CYP26A1. Further, this indicates that the definitive hematopoiesis derived from human pluripotent stem cells is retinoic acid-independent.

In contrast, when the mesodermal KDR+ CXCR4+ population is isolated and cultured in a similar fashion as above, give rise to a CD34+ population. However, this population completely lacked any hematopoietic potential. Similarly, if the ALDH inhibitor DEAB is added, a CD34+ population is obtained, but completely lacked any hematopoietic potential. Critically, if a RA signaling agent, such as the RA precursor, retinol, is added on day 3 of differentiation to these KDR+ CXCR4+ cells, a CD34+ HE population can be obtained by day 6, 7, or 8 of differentiation, between about day 6 and day 14, or between day 8 and day 12. This CD34+ HE population is capable of erythro-myeloid-lymphoid multilineage hematopoiesis. Therefore, this CD34+HE is representative of RA-dependent definitive hematopoiesis, and is derived from KDR+ CXCR4+ mesodermal cells that express ALDH1A2.

This RA-dependent HE can be highly dependent on the correct temporal application of RA signaling. When applied at day 3 of differentiation to isolated KDR+ CXCR4+ mesoderm, RA-dependent HE is specified. However, if RA signaling is applied 1 or 2 days later (day 4 or 5 of differentiation), CD34+ cells are obtained, but these CD34+ cells completely lack hematopoietic potential. Therefore, there is a critical stage-specific role for RA signaling in the specification of this HE population.

Obtaining this RA-dependent HE does not require FACS isolation of KDR+ CXCR4+ mesoderm. If RA signaling is applied to bulk differentiation cultures on day 3 of differentiation, which possess a KDR+ CXCR4+ subset, these cells will respond to the RA agonist and specify a CD34+ HE population that persists from days 8-16 of differentiation.

To-date, there have been many published attempts to identify a RA-dependent HE from hPSCs. However, it is believed that none have elegantly manipulated BMP4, WNT, ACTIVIN/NODAL, and RA in the correct temporal order. In contrast, disclosed herein is a unique, stage-specific method to generate RA-dependent definitive hematopoietic progenitors from hPSCs. Further, the mesodermal population that gives rise to these CD34+ hematopoietic progenitors have been identified.

The present disclosure provides for a method to obtain retinoic acid-dependent hematopoietic progenitors from human pluripotent stem cells.

BMP4, then bFGF, then WNT, and ACTIVIN/NODAL, followed by retinoic acid (RA) can be used to derive different population of progenitors from embryonic stem cells and induced pluripotent stem cells (collectively, human pluripotent stem cells, hPSCs).

It is presently believed no one has successfully derived RA-dependent hemogenic endothelial cells capable of hematopoiesis. These HECs can be capable of being used for replacement blood products (e.g., universal stem cells).

The present disclosure provides for the generation of RA-dependent hematopoietic progenitors from hPSCs. The method includes sequential, stage-specific manipulation of BMP4, bFGF, WNT, and RA signaling.

Described herein is the ability to derive RA-dependent hematopoietic progenitors from hPSCs. The temporal signaling (e.g., day 3 of differentiation) was discovered to be important—if RA signaling is applied 1 or 2 days later, similar cells are obtained (i.e., same markers expressed) but do not have hematopoietic potential. The differentiation protocol, as described herein, has yielded subsets of progenitor cells capable of multi-lineage hematopoiesis.

### (d) Hematopoietic Maturation

The hematopoietic progenitor cells obtained from the hematopoietic specification step may be further cultured in a maturation medium to be differentiated into specific types of blood cells (e.g., red blood cells, platelets, neutrophils, megakaryocytes, etc.) in vitro or ex vivo before administration to a subject. The hematopoietic progenitor cells can be differentiated into specific types of blood cells using any methods described herein or known in the art. For example, any of the growth factors known to promote cell differentiation into specific type of hematopoietic cells described herein or known in the art can be used. In particular, the following references describe methods for differentiation of hematopoietic progenitor cells that can be used for differentiation of the hematopoietic progenitor cells: Zeuner et al., 2012, Stem Cells 30:1587-96; Ebihara et al., 2012, Int J Hematol 95:610-6; Takayama & Eto, 2012, Cell Mol Life Sci 69:3419-28; Takayama & Eto, 2012, Methods Mol Biol 788:205-17; and Kimbrel & Lu, 2011, Stem Cells Int., March 8; doi:10.4061/2011/273076. In one embodiment, the hematopoietic progenitor cells are differentiated into red blood cells; such red blood cells can be administered to a subject. In one embodiment, the hematopoietic progenitor cells are differentiated into neutrophils; and such neutrophils can be administered to a subject. In one embodiment, the hematopoietic progenitor cells are differentiated into platelets; and such platelets can be administered to a patient. In certain embodiments, hematopoietic progenitor cells are generated in accordance with the methods described herein (optionally, gene-corrected), differentiated into specific types of hematopoietic cells (e.g., red blood cells, neutrophils or platelets), and the differentiated cells produced from the hematopoietic progenitor cells are administered to a subject.

As will be apparent, methods and products as described herein with respect to the hematopoietic progenitor cells will also apply to the differentiated cells produced from the hematopoietic progenitor cells, unless the context would indicate otherwise to one skilled in the art.

### (e) Genetic Modification of Pluripotent Stem Cells or Hematopoietic Progenitor Cells

In some embodiments, the pluripotent stem cells used in the in vitro culturing system disclosed herein or the hematopoietic progenitor cells produced by the same may be genetically modified such that a gene of interest is modulated. Accordingly, the present disclosure also provides methods of preparing such genetically modified pluripotent stem cells or hematopoietic progenitor cells. In some embodiments, the gene of interest is disrupted. As used herein, the term “a disrupted gene” refers to a gene containing one or more mutations (e.g., insertion, deletion, or nucleotide substitution, etc.) relative to the wild-type counterpart so as to substantially reduce or completely eliminate the activity of the encoded gene product. The one or more mutations may be located in a non-coding region, for example, a promoter region, a regulatory region that regulates transcription or translation; or an intron region. Alternatively, the one or more mutations may be located in a coding region (e.g., in an exon). In some instances, the disrupted gene does not express or express a substantially reduced level of the encoded protein. In other instances, the disrupted gene expresses the encoded protein in a mutated form, which is either not functional or has substantially reduced activity. In some embodiments, a disrupted gene does not express (e.g., encode) a functional protein.

Techniques such as CRISPR (particularly using Cas9 and guide RNA), editing with zinc finger nucleases (ZFNs) and transcription activator-like effector nucleases (TALENs) may be used to produce the genetically engineered pluripotent stem cells.

‘Genetic modification’, ‘genome editing’, or ‘genomic editing’, or ‘genetic editing’, as used interchangeably herein, is a type of genetic engineering in which DNA is inserted, deleted, and/or replaced in the genome of a targeted cell. Targeted genome modification (interchangeable with “targeted genomic editing” or “targeted genetic editing”) enables insertion, deletion, and/or substitution at pre-selected sites in the genome. When an endogenous sequence is deleted at the insertion site during targeted editing, an endogenous gene comprising the affected sequence may be knocked-out or knocked-down due to the sequence deletion. In another aspect, an endogenous gene may be modified by introducing a change in an endogenous gene codon, wherein the modification introduces an amino acid change in the gene product or introduction of a stop codon. Therefore, targeted modification may also be used to disrupt endogenous gene expression with precision. Similarly used herein is the term “targeted integration,” referring to a process involving insertion of one or more exogenous sequences, with or without deletion of an endogenous sequence at the insertion site. In comparison, randomly integrated genes are subject to position effects and silencing, making their expression unreliable and unpredictable. For example, centromeres and sub-telomeric regions are particularly prone to transgene silencing. Reciprocally, newly integrated genes may affect the surrounding endogenous genes and chromatin, potentially altering cell behavior or favoring cellular transformation. Therefore, inserting exogenous DNA in a pre-selected locus such as a safe harbor locus, or genomic safe harbor (GSH) is important for safety, efficiency, copy number control, and for reliable gene response control.

Targeted modification can be achieved either through a nuclease-independent approach, or through a nuclease-dependent approach. In the nuclease-independent targeted editing approach, homologous recombination is guided by homologous sequences flanking an exogenous polynucleotide to be inserted, through the enzymatic machinery of the host cell.

Alternatively, targeted modification could be achieved with higher frequency through specific introduction of double strand breaks (DSBs) by specific rare-cutting endonucleases. Such nuclease-dependent targeted editing utilizes DNA repair mechanisms including non-homologous end joining (NHEJ), which occurs in response to DSBs. Without a donor vector containing exogenous genetic material, the NHEJ often leads to random insertions or deletions (in/dels) of a small number of endogenous nucleotides. In comparison, when a donor vector containing exogenous genetic material flanked by a pair of homology arms is present, the exogenous genetic material can be introduced into the genome during homology directed repair (HDR) by homologous recombination, resulting in a “targeted integration.”

In some embodiments, non-limiting examples of targeted nucleases include naturally occurring and recombinant nucleases; CRISPR related nucleases from families including cas, cpf, cse, csy, csn, csd, cst, csh, csa, csm, and cmr; restriction endonucleases; meganucleases; homing endonucleases, and the like.

In an exemplary embodiment, the CRISPR/Cas9 gene editing technology is used for producing the genetically engineered pluripotent stem cells. Typically, CRISPR/Cas9 requires two major components: (1) a Cas9 endonuclease and (2) the crRNA-tracrRNA complex. When co-expressed, the two components form a complex that is recruited to a target DNA sequence comprising PAM and a seeding region near PAM. The crRNA and tracrRNA can be combined to form a chimeric guide RNA (gRNA) to guide Cas9 to target selected sequences. These two components can then be delivered to mammalian cells via transfection or transduction. Any known CRISPR/Cas9 methods can be used in the methods disclosed herein. See also Examples below.

Besides the CRISPR method disclosed herein, additional gene editing methods as known in the art can also be used in making the genetically engineered T cells disclosed herein. Some examples include gene editing approaching involve zinc finger nuclease (ZFN), transcription activator-like effector nucleases (TALEN), restriction endonucleases, meganucleases homing endonucleases, and the like.

ZFNs are targeted nucleases comprising a nuclease fused to a zinc finger DNA binding domain (ZFBD), which is a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but not limited to, C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A selected zinc finger domain is a domain not found in nature whose production results primarily from an empirical process such as phage display, interaction trap or hybrid selection. ZFNs are described in greater detail in U.S. Pat. Nos. 7,888,121 and 7,972,854. The most recognized example of a ZFN is a fusion of the Fokl nuclease with a zinc finger DNA binding domain.

A TALEN is a targeted nuclease comprising a nuclease fused to a TAL effector DNA binding domain. A “transcription activator-like effector DNA binding domain”, “TAL effector DNA binding domain”, or “TALE DNA binding domain” is a polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in US Patent Application No. 2011/0145940. The most recognized example of a TALEN in the art is a fusion polypeptide of the Fokl nuclease to a TAL effector DNA binding domain.

Additional examples of targeted nucleases suitable for use as provided herein include, but are not limited to, Bxb1, phiC31, R4, PhiBT1, and Wp/SPBc/TP901-1, whether used individually or in combination.

Any of the gene editing nucleases disclosed herein may be delivered using a vector system, including, but not limited to, plasmid vectors, DNA minicircles, retroviral vectors, lentiviral vectors, adenovirus vectors, poxvirus vectors; herpesvirus vectors and adeno-associated virus vectors, and combinations thereof.

Conventional viral and non-viral based gene transfer methods can be used to introduce nucleic acids encoding nucleases and donor templates in cells (e.g., T cells). Non-viral vector delivery systems include DNA plasmids, DNA minicircles, naked nucleic acid, and nucleic acid complexed with a delivery vehicle such as a liposome or poloxamer. Viral vector delivery systems include DNA and RNA viruses, which have either episomal or integrated genomes after delivery to the cell.

Methods of non-viral delivery of nucleic acids include electroporation, lipofection, microinjection, biolistics, virosomes, liposomes, immunoliposomes, polycation or lipid:nucleic acid conjugates, naked DNA, naked RNA, capped RNA, artificial virions, and agent-enhanced uptake of DNA. Sonoporation using, e.g., the Sonitron 2000 system (Rich-Mar) can also be used for delivery of nucleic acids.

### II. Methods

Any of the hematopoietic progenitor cells produced by the methods of various aspects described herein (e.g., the methods of Section I) can be used in different applications where hematopoietic progenitor cells are required. Such uses of hematopoietic progenitor cells are also within the scope of the present disclosure.

In some embodiments, the hematopoietic progenitor cells are obtained from cells derived from a subject to whom the hematopoietic progenitor cells are to be administered. In such embodiments, the embryonic hematopoietic stem cells can be derived from ESC, iPSC or reprogrammed non-pluripotent cells derived from the subject to whom the hematopoietic progenitor cells or cells derived therefrom are to be administered. In a specific embodiment, adult cells can be obtained from a subject, such cells can be reprogrammed to iPSC and then hematopoietic progenitor cells of the disclosure. In specific embodiments, hematopoietic progenitor cells are derived from cells of a patient with a genetic disorder associated with a gene having a sequence detect, and such hematopoietic progenitor cells are genetically engineered to correct the sequence defect before administration to the subject. In one embodiment, hematopoietic progenitor cells are derived from cells of a subject with a genetic disorder associated with a gene having a sequence defect, and such hematopoietic progenitor cells are genetically engineered to correct the sequence defect, and the genetically engineered hematopoietic progenitor cells or cells derived therefrom are administered to the patient.

Once generated the hematopoietic progenitor cells or cells differentiated therefrom can be cryopreserved in accordance with the methods described below or known in the art.

In one embodiment, a hematopoietic progenitor cell population can be divided and frozen in one or more bags (or units). In another embodiment, two or more hematopoietic progenitor cell populations can be pooled, divided into separate aliquots, and each aliquot is frozen. In a preferred embodiment, a maximum of approximately 4 billion nucleated cells is frozen in a single bag. In a preferred embodiment, the hematopoietic progenitor cells are fresh, i.e., they have not been previously frozen prior to expansion or cryopreservation. The terms “frozen/freezing” and “cryopreserved/cryopreserving” are used interchangeably in the present application. Cryopreservation can be by any method in known in the art that freezes cells in viable form. The freezing of cells is ordinarily destructive. On cooling, water within the cell freezes. Injury then occurs by osmotic effects on the cell membrane, cell dehydration, solute concentration, and ice crystal formation. As ice forms outside the cell, available water is removed from solution and withdrawn from the cell, causing osmotic dehydration and raised solute concentration which eventually destroys the cell. For a discussion, see Mazur, P., 1977, Cryobiology 14:251-272.

These injurious effects can be circumvented by (a) use of a cryoprotective agent, (b) control of the freezing rate, and (c) storage at a temperature sufficiently low to minimize degradative reactions.

Cryoprotective agents which can be used include but are not limited to dimethyl sulfoxide (DMSO) (Lovelock and Bishop, 1959, Nature 183:1394-1395; Ashwood-Smith, 1961, Nature 190:1204-1205), glycerol, polyvinylpyrrolidine (Rinfret, 1960, Ann, N.Y. Acad. Sci. 85:576), polyethylene glycol (Sloviter and Ravdin, 1962, Nature 196:548), albumin, dextran, sucrose, ethylene glycol, i-erythritol, D-ribitol, D-mannitol (Rowe et al., 1962, Fed. Proc. 21:157), D-sorbitol, i-inositol, D-lactose, choline chloride (Bender et al., 1960, J. Appl. Physiol. 15:520), amino acids (Phan The Tran and Bender, 1960, Exp. Cell Res. 20:651), methanol, acetamide, glycerol monoacetate (Lovelock, 1954, Biochem. J. 56:265), and inorganic salts (Phan The Tran and Bender, 1960, Proc. Soc. Exp. Biol. Med. 104:388; Phan The Tran and Bender, 1961, in Radiobiology, Proceedings of the Third Australian Conference on Radiobiology, Ilbery ed., Butterworth, London, p. 59). In a preferred embodiment, DMSO is used, a liquid which is nontoxic to cells in low concentration. Being a small molecule, DMSO freely permeates the cell and protects intracellular organelles by combining with water to modify its freezability and prevent damage from ice formation. Addition of plasma (e.g., to a concentration of 20-25%) can augment the protective effect of DMSO. After addition of DMSO, cells should be kept at 0° C. until freezing, since DMSO concentrations of about 1% are toxic at temperatures above 4° C.

A controlled slow cooling rate can be critical. Different cryoprotective agents (Rapatz et al., 1968, Cryobiology 5(1):18-25) and different cell types have different optimal cooling rates (see e.g., Rowe and Rinfret, 1962, Blood 20:636; Rowe, 1966, Cryobiology 3(1):12-18; Lewis, et al., 1967, Transfusion 7(1):17-32; and Mazur, 1970, Science 168:939-949 for effects of cooling velocity on survival of marrow-stem cells and on their transplantation potential). The heat of fusion phase where water turns to ice should be minimal. The cooling procedure can be carried out by use of e.g., a programmable freezing device or a methanol bath procedure.

Programmable freezing apparatuses allow determination of optimal cooling rates and facilitate standard reproducible cooling. Programmable controlled-rate freezers such as Cryomed or Planar permit tuning of the freezing regimen to the desired cooling rate curve. For example, for marrow cells in 10% DMSO and 20% plasma, the optimal rate is 1° to 3° C./minute from 0° C. to −80° C. In a preferred embodiment, this cooling rate can be used for CB cells. The container holding the cells must be stable at cryogenic temperatures and allow for rapid heat transfer for effective control of both freezing and thawing. Sealed plastic vials (e.g., Nunc, Wheaton cryules) or glass ampules can be used for multiple small amounts (1-2 ml), while larger volumes (100-200 ml) can be frozen in polyolefin bags (e.g., Delmed) held between metal plates for better heat transfer during cooling. Bags of bone marrow cells have been successfully frozen by placing them in −80° C. freezers which, fortuitously, gives a cooling rate of approximately 3° C./minute).

In an alternative embodiment, the methanol bath method of cooling can be used. The methanol bath method is well-suited to routine cryopreservation of multiple small items on a large scale. The method does not require manual control of the freezing rate nor a recorder to monitor the rate. In a preferred embodiment, DMSO-treated cells are pre-cooled on ice and transferred to a tray containing chilled methanol which is placed, in turn, in a mechanical refrigerator (e.g., Harris or Revco) at −80° C. Thermocouple measurements of the methanol bath and the samples indicate the desired cooling rate of 1° to 3° C./minute. After at least two hours, the specimens have reached a temperature of −80° C. and can be placed directly into liquid nitrogen (−196° C.) for permanent storage.

After thorough freezing, the hematopoietic progenitor cells can be rapidly transferred to a long-term cryogenic storage vessel. In a preferred embodiment, samples can be cryogenically stored in liquid nitrogen (−196° C.) or its vapor (−165° C.). Such storage is greatly facilitated by the availability of highly efficient liquid nitrogen refrigerators, which resemble large Thermos containers with an extremely low vacuum and internal super insulation, such that heat leakage and nitrogen losses are kept to an absolute minimum.

Suitable racking systems are commercially available and can be used for cataloguing, storage, and retrieval of individual specimens.

Considerations and procedures for the manipulation, cryopreservation, and long-term storage of the hematopoietic stem cells, particularly from bone marrow or peripheral blood (e.g., mobilized peripheral blood), which are also largely applicable to the Expanded eHSC can be found, for example, in the following references, incorporated by reference herein: Gorin, 1986, Clinics In Haematology 15(1):19-48; Bone-Marrow Conservation, Culture and Transplantation, Proceedings of a Panel, Moscow, Jul. 22-26, 1968, International Atomic Energy Agency, Vienna, pp. 107-186.

Other methods of cryopreservation of viable cells, or modifications thereof, are available and envisioned for use (e.g., cold metal-mirror techniques; Livesey and Linner, 1987, Nature 327:255; Linner et al., 1986, J. Histochem. Cytochem. 34(9):1123-1135; see also U.S. Pat. No. 4,199,022 by Senkan et al., U.S. Pat. No. 3,753,357 by Schwartz, U.S. Pat. No. 4,559,298 by Fahy).

In other embodiments, generated hematopoietic progenitor cells or cells derived therefrom are preserved by freeze-drying (see Simione, 1992, J. Parenter. Sci. Technol. 46(6):226-32).

Following cryopreservation, frozen isolated hematopoietic progenitor cells can be thawed in accordance with the methods described below or known in the art.

Frozen cells are preferably thawed quickly (e.g., in a water bath maintained at 37°−41° C.) and chilled immediately upon thawing. In a specific embodiment, the vial containing the frozen cells can be immersed up to its neck in a warm water bath; gentle rotation will ensure mixing of the cell suspension as it thaws and increase heat transfer from the warm water to the internal ice mass. As soon as the ice has completely melted, the vial can be immediately placed in ice.

In an embodiment of the disclosure, the hematopoietic progenitor cell sample as thawed, or a portion thereof, can be infused for providing hematopoietic function in a human patient in need thereof. Several procedures, relating to processing of the thawed cells are available, and can be employed if deemed desirable.

It may be desirable to treat the cells in order to prevent cellular clumping upon thawing. To prevent clumping, various procedures can be used, including but not limited to, the addition before and/or after freezing of DNase (Spitzer et al., 1980, Cancer 45:3075-3085), low molecular weight dextran and citrate, hydroxyethyl starch (Stiff et al., 1983, Cryobiology 20:17-24), etc.

The cryoprotective agent, if toxic in humans, should be removed prior to therapeutic use of the thawed hematopoietic progenitor cells. In an embodiment employing DMSO as the cryopreservative, it is preferable to omit this step in order to avoid cell loss, since DMSO has no serious toxicity. However, where removal of the cryoprotective agent is desired, the removal is preferably accomplished upon thawing.

One way in which to remove the cryoprotective agent is by dilution to an insignificant concentration. This can be accomplished by addition of medium, followed by, if necessary, one or more cycles of centrifugation to pellet cells, removal of the supernatant, and resuspension of the cells. For example, intracellular DMSO in the thawed cells can be reduced to a level (less than 1%) that will not adversely affect the recovered cells. This is preferably done slowly to minimize potentially damaging osmotic gradients that occur during DMSO removal.

After removal of the cryoprotective agent, cell count (e.g., by use of a hemocytometer) and viability testing (e.g., by trypan blue exclusion; Kuchler, 1977, Biochemical Methods in Cell Culture and Virology, Dowden, Hutchinson & Ross, Stroudsburg, Pa., pp. 18-19; 1964, Methods in Medical Research, Eisen et al., eds., Vol. 10, Year Book Medical Publishers, Inc., Chicago, pp. 39-47) can be done to confirm cell survival. The percentage of viable antigen (e.g., CD34) positive cells in a sample can be determined by calculating the number of antigen positive cells that exclude 7-AAD (or other suitable dye excluded by viable cells) in an aliquot of the sample, divided by the total number of nucleated cells (TNC) (both viable and non-viable) in the aliquot of the sample. The number of viable antigen positive cells in the sample can be then determined by multiplying the percentage of viable antigen positive cells by TNC of the sample.

Optionally, the hematopoietic progenitor cell sample can undergo HLA typing either prior to cryopreservation and/or after cryopreservation and thawing. HLA typing can be performed using serological methods with antibodies specific for identified HLA antigens, or using DNA-based methods for detecting polymorphisms in the HLA antigen-encoding genes for typing HLA alleles. In a specific embodiment, HLA typing can be performed at intermediate resolution using a sequence specific oligonucleotide probe method for HLA-A and HLA-B or at high resolution using a sequence based typing method (allele typing) for HLA-DRB 1.

The hematopoietic progenitor cells, whether recombinantly expressing a desired gene, having been corrected for a defective gene, or not, can be administered into a human subject in need thereof for hematopoietic function for the treatment of disease or injury or for gene therapy by any method known in the art which is appropriate for the hematopoietic progenitor cells and the transplant site. Preferably, the hematopoietic progenitor cells or cells derived therefrom are transplanted (infused) intravenously. In one embodiment, the hematopoietic progenitor cells differentiate into cells of the myeloid lineage in the patient. In another embodiment, the hematopoietic progenitor cells differentiate into cells of the lymphoid lineage in the patient.

In one embodiment, the transplantation of the hematopoietic progenitor cells is autologous. In such embodiments, before expansion, cells are isolated from tissues of a subject to whom hematopoietic progenitor cells are to be administered, reprogrammed to iPSC and then hematopoietic progenitor cells, or directly reprogrammed to hematopoietic progenitor cells and, optionally, gene-corrected as described above. In other embodiments, the transplantation of the hematopoietic progenitor cells is non-autologous. In some of these embodiments, the transplantation of the hematopoietic progenitor cells is allogeneic. For non-autologous transplantation, the recipient can be given an immunosuppressive drug to reduce the risk of rejection of the transplanted cells. In some embodiments, the transplantation of the hematopoietic progenitor cell is syngeneic.

In specific embodiments, hematopoietic progenitor cells or cells derived therefrom are administered to a subject with a hematopoietic disorder as described herein.

In some embodiments, the hematopoietic progenitor cell sample that is administered to the subject has been cryopreserved and thawed prior to administration. In other embodiments, the hematopoietic progenitor cell sample that is administered to the subject is fresh, i.e., it has not been cryopreserved prior to administration.

In certain embodiments, the hematopoietic progenitor cells are intended to provide short-term engraftment. Short-term engraftment usually refers to engraftment that lasts for up to a few days to few weeks, preferably 4 weeks, post-transplantation of the hematopoietic progenitor cell. In some embodiments, the hematopoietic progenitor cells are effective to provide engraftment 1, 2, 3, 4, 5, 6, 7, 8, 9, 10 days; or 1, 2, 3, 4 weeks after administration of the hematopoietic progenitor cells to a subject (e.g., a human patient). In other embodiments, the hematopoietic progenitor cells are intended to provide long-term engraftment. Long-term engraftment usually refers to engraftment that is present months to years post-transplantation of the hematopoietic progenitor cells. In some embodiments, the hematopoietic progenitor cells are effective to provide engraftment when assayed at 8, 9, 10 weeks; 2, 3, 4, 5, 6, 7, 8, 9, 10, 11, 12 months for more than 2, 3, 4, 5, 6, 7, 8, 9, 10, 11, 12 months); or 1, 2, 3, 4, 5 years (or more than 1, 2, 3, 4, 5 years) after administration of the hematopoietic progenitor cells to a subject. In some embodiments, the hematopoietic progenitor cells are intended to provide both short-term and long-term engraftment. In certain embodiments, the hematopoietic progenitor cells provide short-term and/or long-term engraftment in a patient, preferably, a human.

In some embodiments, the hematopoietic progenitor cells are effective to provide engraftment when assayed at 1, 2, 3, 4, 5, 6, 7, 8, 9, 10 days (or more than 1, 2, 3, 4, 5, 6, 7, 8, 9, 10 days); 1, 2, 3, 4, 5, 6, 7, 8, 9, 10 weeks (or more than 1, 2, 3, 4, 5, 6, 7, 8, 9, 10 weeks); 1; 2, 3, 4, 5, 6, 7, 8, 9, 10, 11, 12 months (or more than 1, 2, 3, 4, 5, 6, 7, 8, 9, 10, 11, 12 months); or 1, 2, 3, 4, 5 years (or more than 1, 2, 3, 4, 5 years) after administration of the hematopoietic progenitor cells to a subject (e.g., a human patient). In other embodiments, the hematopoietic progenitor cells are effective to provide engraftment when assayed within 1, 2, 3, 4, 5, 6, 7, 8, 9, 10 days (or less than 1, 2, 3, 4, 5, 6, 7, 8, 9, 10 days); 1, 2, 3, 4, 5, 6, 7, 8, 9, 10 weeks for less than 1, 2, 3, 4, 5, 6, 7, 8, 9, 10 weeks); or 1; 2, 3, 4, 5, 6, 7, 8, 9, 10, 11, 12 months (or less than 1, 2, 3, 4, 5, 6, 7, 8, 9, 10, 11, 12 months) after administration of the hematopoietic progenitor cells to a subject (e.g., a human patient). In specific embodiments, the hematopoietic progenitor cells are effective to provide engraftment when assayed within 10 days, 2 weeks, 3 weeks, 4 weeks, 6 weeks, 6 weeks, or 13 weeks after administration of the hematopoietic progenitor cells to a subject (e.g., a human patient).

Suitable methods of administration of the hematopoietic progenitor cells are encompassed by the present disclosure. The hematopoietic progenitor cells populations can be administered by any convenient route, for example by infusion or bolus injection, and may be administered together with other biologically active agents. Administration can be systemic or local.

The titer of the hematopoietic progenitor cells administered which will be effective in the treatment of a particular disorder or condition will depend on the nature of the disorder or condition, and can be determined by standard clinical techniques. In addition, in vitro and in vivo assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. In specific embodiments, suitable dosages of the hematopoietic progenitor cells for administration are generally about at least 5×10⁶, 10⁷, 5×10⁷, 75×10⁶, 10⁷, 5×10⁷, 10⁸, 5×10⁸, 1×10⁹, 5×10⁹, 1×10¹⁰, 5×10¹⁰, 1×10¹¹, 5×10¹¹ or 10¹² CD34+ cells per kilogram patient weight, and most preferably about 10⁷ to about 10¹² CD34+ cells per kilogram patient weight, and can be administered to a patient once, twice, three or more times with intervals as often as needed. In a specific embodiment, a single hematopoietic progenitor cells sample provides one or more doses for a single patient. In one specific embodiment, a single hematopoietic progenitor cells sample provides four doses for a single patient.

In certain embodiments, the patient is a human patient, preferably a human patient with a hematopoietic disorder or an immunodeficient human patient.

In a specific embodiment, the hematopoietic progenitor cell population administered to a human patient in need thereof can be a pool of two or more samples derived from a single human. As used herein the terms “patient” and “subject” are used interchangeably.

The disclosure provides methods of treatment by administration to a patient of a pharmaceutical (therapeutic) composition comprising a therapeutically effective amount of recombinant or non-recombinant hematopoietic progenitor cells produced by the methods of the present invention as described herein above.

The present disclosure provides pharmaceutical compositions. Such compositions comprise a therapeutically effective amount of the hematopoietic progenitor cells or cells derived therefrom, and a pharmaceutically acceptable carrier or excipient. Such a carrier can be but is not limited to saline, buffered saline, dextrose, water, glycerol, ethanol, and combinations thereof. The carrier and composition preferably are sterile. Suitable pharmaceutical carriers are described in Remington: The Science and Practice of Pharmacy, 21st Edition, David B. Troy, ed., Lippicott Williams & Wilkins (2005), which is incorporated by reference herein in its entirety, and specifically for the material related to pharmaceutical carriers and compositions. The pharmaceutical compositions described herein can be formulated in any manner known in the art.

The formulation should suit the mode of administration. Hematopoietic progenitor cells can be resuspended in a pharmaceutically acceptable medium suitable for administration to a mammalian host. In preferred embodiments, the pharmaceutical composition is acceptable for therapeutic use in humans. The composition, if desired, can also contain pH buffering agents.

The pharmaceutical compositions described herein can be administered via any route known to one skilled in the art to be effective. In a preferred embodiment, the composition is formulated in accordance with routine procedures as a pharmaceutical composition adapted fir intravenous administration to a patient (e.g., a human). Typically, compositions for intravenous administration are solutions in sterile isotonic aqueous buffer. Where necessary, the composition may also include a solubilizing agent and a local anesthetic such as lidocaine to ease pain at the site of the injection.

In specific embodiments, the compositions described herein are formulated for administration to a patient with one or more additional therapeutic active ingredients.

The hematopoietic progenitor cells of the present disclosure can be used to provide hematopoietic function to a patient in need thereof, preferably a human patient. In other embodiments, the patient is a cow, a pig, a horse, a dog, a cat, or any other animal, preferably a mammal.

The patient to whom the hematopoietic progenitor cells are administered is a patient of any age post-birth, e.g., a newborn, an infant, a child or an adult (e.g., a human newborn, a human infant, a human child or a human adult).

In one embodiment, administration of hematopoietic progenitor cells of the invention is for the treatment of immunodeficiency. In a preferred embodiment, administration of hematopoietic progenitor cells of the disclosure is for the treatment of pancytopenia or for the treatment of neutropenia. The immunodeficiency in the patient, for example, pancytopenia or neutropenia, can be the result of an intensive chemotherapy regimen, myeloablative regimen for hematopoietic cell transplantation (HCT), or exposure to acute ionizing radiation. Exemplary chemotherapeutics that can cause prolonged pancytopenia or prolonged neutropenia include, but are not limited to alkylating agents such as cisplatin, carboplatin, and oxaliplatin, mechlorethamine, cyclophosphamide, chlorambucil, and ifosfamide. Other chemotherapeutic agents that can cause prolonged pancytopenia or prolonged neutropenia include azathioprine, mercaptopurine, vinca alkaloids, e.g., vincristine, vinblastine, vinorelbine, vindesine, and taxanes. In particular, a chemotherapy regimen that can cause prolonged pancytopenia or prolonged neutropenia is the administration of clofarabine and Ara-C.

In one embodiment, the patient is in an acquired or induced aplastic state.

The immunodeficiency in the patient also can be caused by exposure to acute ionizing radiation following a nuclear attack, e.g., detonation of a “dirty” bomb in a densely populated area, or by exposure to ionizing radiation due to radiation leakage at a nuclear power plant, or exposure to a source of ionizing radiation, raw uranium ore.

Transplantation of hematopoietic progenitor cells of the invention can be used in the treatment or prevention of hematopoietic disorders and diseases. In one embodiment, the hematopoietic progenitor cells are administered to a patient with a hematopoietic deficiency. In one embodiment, the hematopoietic progenitor cells are used to treat or prevent a hematopoietic disorder or disease characterized by a failure or dysfunction of normal blood cell production and cell maturation. In another embodiment, the hematopoietic progenitor cells are used to treat or prevent a hematopoietic disorder or disease resulting from a hematopoietic malignancy. In yet another embodiment, the hematopoietic progenitor cells are used to treat or prevent a hematopoietic disorder or disease resulting from immunosuppression, particularly immunosuppression in subjects with malignant, solid tumors. In yet another embodiment, the hematopoietic progenitor cells are used to treat or prevent an autoimmune disease affecting the hematopoietic system. In yet another embodiment, the hematopoietic progenitor cells are used to treat or prevent a genetic or congenital hematopoietic disorder or disease.

Examples of particular hematopoietic diseases and disorders which can be treated by the hematopoietic progenitor cells of the disclosure include but are not limited to diseases resulting from a failure or dysfunction of normal blood cell production and maturation. In non-limiting examples, hyperproliferative stem cell disorders, aplastic anemia, pancytopenia, agranulocytosis, thrombocytopenia, red cell aplasia, Blackfan-Diamond syndrome, due to drugs, radiation, or infection Idiopathic II. Hematopoietic malignancies, acute lymphoblastic (lymphocytic) leukemia, chronic lymphocytic leukemia, acute myelogenous leukemia, chronic myelogenous leukemia, acute malignant myelosclerosis, multiple myeloma polycythemia, vera agnogenic myelometaplasia, Waldenstrom's macroglobulinemia, Hodgkin's lymphoma, non-Hodgkin's lymphoma. Immunosuppression in patients with malignant, solid tumors, malignant melanoma, carcinoma of the stomach, ovarian carcinoma, breast carcinoma, small cell lung carcinoma, retinoblastoma, testicular carcinoma, glioblastoma, rhabdomyosarcoma, neuroblastoma, Ewing's sarcoma, lymphoma. Autoimmune diseases, rheumatoid arthritis, diabetes type I, chronic hepatitis, multiple sclerosis, systemic lupus, erythematosus. Genetic (congenital) disorders, anemias, familial aplastic Fanconi's syndrome (Fanconi anemia), Bloom's syndrome, pure red cell aplasia (PRCA), dyskeratosis, congenital Blackfan-Diamond syndrome, congenital dyserythropoietic syndromes. Shwachmann-Diamond syndrome, dihydrofolate reductase deficiencies, formamino transferase deficiency, Lesch-Nyhan syndrome, congenital spherocytosis, congenital elliptocytosis, congenital stomatocytosis, congenital Rh null disease, paroxysmal nocturnal hemoglobinuria, G6PD (glucose-6-phosphate dehydrogenase) variants, 1, 2, 3 pyruvate kinase deficiency, congenital erythropoietin sensitivity deficiency, sickle cell disease, and trait (Sickle cell anemia) thalassemia alpha, beta, gamma, met-hemoglobinemia, congenital disorders of immunity severe combined immunodeficiency disease (SCID), bare lymphocyte syndrome, ionophore-responsive combined immunodeficiency, combined immunodeficiency with a capping abnormality, nucleoside phosphorylase deficiency, granulocyte actin deficiency, infantile agranulocytosis, Gaucher's disease, adenosine deaminase deficiency, Kostmann's syndrome, reticular dysgenesis, congenital leukocyte dysfunction syndrome. Osteopetrosis, myelosclerosis, acquired hemolytic anemias, acquired immunodeficiencies infectious disorders causing primary or secondary immunodeficiencies bacterial infections (e.g., Brucellosis, Listerosis, tuberculosis, leprosy) parasitic infections (e.g., malaria, Leishmaniasis) fungal infections disorders involving disproportions in lymphoid cell sets and impaired immune functions due to aging phagocyte disorders Kostmann's agranulocytosis chronic granulomatous disease Chediak-Higachi syndrome neutrophil actin deficiency neutrophil membrane GP-180 deficiency metabolic storage diseases mucopolysaccharidoses mucolipidoses miscellaneous disorders involving immune mechanisms Wiskott-Aldrich Syndrome α1-antitrypsin deficiency.

In one embodiment, the hematopoietic progenitor cells are administered to a patient with a hematopoietic deficiency. Hematopoietic deficiencies whose treatment with the hematopoietic progenitor cells of the disclosure is encompassed by the methods of the disclosure include but are not limited to decreased levels of either myeloid, erythroid, lymphoid, or megakaryocyte cells of the hematopoietic system or combinations thereof. In one embodiment, the hematopoietic progenitor cells are administered prenatally to a fetus diagnosed with hematopoietic deficiency.

Among conditions susceptible to treatment with the hematopoietic progenitor cells of the present disclosure is leukopenia, a reduction in the number of circulating leukocytes (white cells) in the peripheral blood. Leukopenia may be induced by exposure to certain viruses or to radiation. It is often a side effect of various forms of cancer therapy, e.g., exposure to chemotherapeutic drugs, radiation and of infection or hemorrhage.

hematopoietic progenitor cells also can be used in the treatment or prevention of neutropenia and, for example, in the treatment of such conditions as aplastic anemia, cyclic neutropenia, idiopathic neutropenia, Chediak-Higashi syndrome, systemic lupus erythematosus (SLE), leukemia, myelodysplastic syndrome, myelofibrosis, thrombocytopenia. Severe thrombocytopenia may result from genetic defects such as Fanconi's Anemia, Wiscott-Aldrich, or May-Hegglin syndromes and from chemotherapy and/or radiation therapy or cancer. Acquired thrombocytopenia may result from auto- or allo-antibodies as in Immune Thrombocytopenia Purpura, Systemic Lupus Erythromatosis, hemolytic anemia, or fetal maternal incompatibility. In addition, splenomegaly, disseminated intravascular coagulation, thrombotic thrombocytopenic purpura, infection or prosthetic heart valves may result in thrombocytopenia. Thrombocytopenia may also result from marrow invasion by carcinoma, lymphoma, leukemia or fibrosis.

Many drugs may cause bone marrow suppression or hematopoietic deficiencies. Examples of such drugs are AZT, DDI, alkylating agents and anti-metabolites used in chemotherapy, antibiotics such as chloramphenicol, penicillin, gancyclovir, daunomycin and sulfa drugs, phenothiazones, tranquilizers such as meprobamate, analgesics such as aminopyrine and dipyrone, anticonvulsants such as phenytoin or carbamazepine, antithyroids such as propylthiouracil and methimazole and diuretics. Transplantation of the hematopoietic progenitor cells can be used in preventing or treating the bone marrow suppression or hematopoietic deficiencies which often occur in subjects treated with these drugs.

Hematopoietic deficiencies may also occur as a result of viral, microbial or parasitic infections and as a result of treatment for renal disease or renal failure, e.g., dialysis. Transplantation of the hematopoietic progenitor cell populations may be useful in treating such hematopoietic deficiency.

Various immunodeficiencies, e.g., in T and/or B lymphocytes, or immune disorders, e.g., rheumatoid arthritis, may also be beneficially affected by treatment with the hematopoietic progenitor cells. Immunodeficiencies may be the result of viral infections (including but not limited to HIVI, HIVII, HTLVI, HTLVII, HTLVIII), severe exposure to radiation, cancer therapy or the result of other medical treatment.

In specific embodiments, the hematopoietic progenitor cells are used for the treatment of multiple myeloma, non-Hodgkin's lymphoma, Hodgkin's disease, neuroblastoma, germ cell tumors, autoimmune disorder (e.g., Systemic lupus erythematosus (SLE) or systemic sclerosis), amyloidosis, acute myeloid leukemia, acute lymphoblastic leukemia, chronic myeloid leukemia, chronic lymphocytic leukemia, myeloproliferative disorder, myelodysplastic syndrome, aplastic anemia, pure red cell aplasia, paroxysmal nocturnal hemoglobinuria, Fanconi anemia, Thalassemia major, Sickle cell anemia, Severe combined immunodeficiency (SCID), Wiskott-Aldrich syndrome, Hemophagocytic lymphohistiocytosis (HLH), or inborn errors of metabolism (e.g., mucopolysaccharidosis, Gaucher disease, metachromatic leukodystrophies or adrenoleukodystrophies). In some embodiments, the hematopoietic progenitor cells are used for the treatment of an inherited immunodeficient disease, an autoimmune disease and/or a hematopoietic disorder.

In one embodiment, the hematopoietic progenitor cells are for replenishment of hematopoietic cells in a patient who has undergone chemotherapy or radiation treatment. In a specific embodiment, the hematopoietic progenitor cells are administered to a patient that has undergone chemotherapy or radiation treatment. In a specific embodiment, the hematopoietic progenitor cells are administered to a patient who has HIV (e.g., for replenishment of hematopoietic cells in a patient who has HIV).

In certain embodiments, the hematopoietic progenitor cells are administered into the appropriate region of a patient's body, for example, by injection into the patient's bone marrow.

In some embodiments, the patient to whom the hematopoietic progenitor cells are administered is a bone marrow donor, at risk of depleted bone marrow, or at risk for depleted or limited blood cell levels. In one embodiment, the patient to whom the hematopoietic progenitor cell is administered is a bone marrow donor prior to harvesting of the bone marrow. In one embodiment, the patient to whom the hematopoietic progenitor cell is administered is a bone marrow donor after harvesting of the bone marrow. In one embodiment, the patient to whom the hematopoietic progenitor cell is administered is a recipient of a bone marrow transplant. In one embodiment, the patient to whom the hematopoietic progenitor cell is administered is elderly, has been exposed or is to be exposed to an immune depleting or myeloablative treatment (e.g., chemotherapy, radiation), has a decreased blood cell level, or is at risk of developing a decreased blood cell level as compared to a control blood cell level. In one embodiment, the patient has anemia or is at risk for developing anemia. In one embodiment, the patient has blood loss due to, e.g., trauma, or is at risk for blood loss. The hematopoietic progenitor cell can be administered to a patient, e.g., before, at the same time, or after chemotherapy, radiation therapy or a bone marrow transplant. In specific embodiments, the patient has depleted bone marrow related to, e.g., congenital, genetic or acquired syndrome characterized by bone marrow loss or depleted bone marrow. In one embodiment, the patient is in need of hematopoiesis.

In some embodiments, the methods and cells produced from the same as disclosed herein can be used, for example, to advance therapeutic discovery. Accordingly, provided herein include a method of screening for an agent for treating a hematopoietic disease or determining the effect of a candidate agent on hematopoietic disease or disorder are also provided herein.

The candidate agents can be selected from the group consisting of proteins, peptides, nucleic acids (e.g., but not limited to, siRNA, anti-miRs, antisense oligonucleotides, and ribozymes), small molecules, nutrients (lipid precursors), and a combination of two or more thereof.

When introducing elements of the present disclosure or the preferred aspects(s) thereof, the articles “a,” “an,” “the,” and “said” are intended to mean that there are one or more of the elements. The terms “comprising,” “including,” and “having” are intended to be inclusive and mean that there may be additional elements other than the listed elements.

As used herein, the following definitions shall apply unless otherwise indicated. For purposes of this invention, the chemical elements are identified in accordance with the Periodic Table of the Elements, CAS version, and the Handbook of Chemistry and Physics, 75^{th} Ed. 1994. Additionally, general principles of organic chemistry are described in “Organic Chemistry,” Thomas Sorrell, University Science Books, Sausalito: 1999, and “March's Advanced Organic Chemistry,” 5^{th} Ed., Smith, M. B. and March, J., eds. John Wiley & Sons, New York: 2001, the entire contents of which are hereby incorporated by reference.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds.(1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Retinoic Acid-Dependent Definitive Hematopoietic Progenitor from Human Pluripotent Stem Cells

The goal of this study was to develop a basis for WNT- and RA-mediated definitive hematopoietic specification and resolve the role of RA in extra-embryonic and intra-embryonic human hematopoietic development. During hematopoietic development, there are at least two distinct anatomical sites of blood cell generation. The first, the extra-embryonic yolk sac, gives rise to multiple hematopoietic programs, such as primitive hematopoiesis, the erythro-myeloid progenitor (EMP), and the lympho-myeloid progenitor (LMPP). While the embryo proper also generates similar transient progenitors, it is distinguished by its unique ability to also give rise to the hematopoietic stem cell (HSC). Central to all these programs is a specialized embryonic hematopoietic progenitor population known as hemogenic endothelium (HE), which is characterized by its unique capacity to undergo an endothelial-to-hematopoietic transition (EHT) to generate hematopoietic progeny. The existence of these different programs, and by extension, functionally distinct HE populations, has contributed to difficulties in understanding the physiological relevance of human pluripotent stem cell (hPSC)-derived hematopoiesis. This is because, as until recently, hPSC differentiation methods could not discriminate between the progenitors of these various programs. However, recent work has demonstrated it is now possible to independently derive pure populations of either extra-embryonic-like or intra-embryonic-like HE through stage-specific manipulation of WNT signaling, and that these populations can be distinguished by differential HOXA expression.

Aside from WNT, these directed differentiation strategies also elegantly employ other signal pathways necessary for hematopoietic development, such as BMP, VEGF, and NOTCH, recapitulating that observed in the vertebrate embryo. However, one critical regulator of intra-embryonic HE development, retinoic acid (RA), has confounded all these aforementioned studies, as they describe methods that give rise to definitive hematopoiesis in an RA-independent manner. Studies in mice have clearly demonstrated that RA is essential for HSC emergence. However, efforts at manipulating RA on hPSC-derived HE have yielded no functional improvements. As such, the identification of an RA-dependent hemogenic precursor has remained elusive.

The present examples provide the identification of an hPSC-derived progenitor population that is uniquely dependent on stage-specific RA signaling. In turn, this resultant HE is functionally and transcriptionally similar to HE found in the human embryo. Further, this work refines the understanding of human hematopoietic development, and suggests a complex series of “waves” of HE, each with a distinct ontogenic origin, with correspondingly different gene expression and functional potentials. Thus, a tractable system which enables the study of the mechanism(s) regulating human definitive hematopoietic specification has now been defined. In particular, the work described herein provides methods for generating physiologically relevant definitive hematopoietic progenitors from hPSCs and, for the first time, provides access to an RA-dependent, human HE. These findings, inter alia, enable the development of novel platforms for identifying the signaling pathways that regulate its specification to HSCs and other hematopoietic lineages which are of great interest for many biomedical applications.

### Methods

### Maintenance and Differentiation of Human ES and iPS Cells

The hESC lines H1 and H9, and human iPSC1 were maintained on irradiated mouse embryonic fibroblasts in hESC media as described previously (Sturgeon, C. M., et al. Nat Biotechnol 32, 554-561, (2014); Thomson, J. A. et al. Science 282, 1145-1147 (1998); Dege, C. et al., J Vis Exp, (2017)). For differentiation, hPSC were cultured on Matrigel-coated plasticware (BD Biosciences, Bedford, MA) for 24 hours, followed by embryoid body (EB) generation, as described previously (Kennedy, M. et al. Cell Rep 2, 1722-1735, (2012); Dege, C. et al. J Vis Exp, (2017); Ditadi, A. et al. Methods 101, 65-72, (2016)). Briefly, hPSCs were dissociated with brief trypsin-EDTA (0.05%) treatment, followed by scraping. Embryoid body (EB) aggregates were resuspended in SFD media34 supplemented with L-glutamine (2 mM), ascorbic acid (1 mM), monothioglycerol (MTG, 4×10⁻⁴ M; Sigma), transferrin (150 μg/mL), and BMP-4 (10 ng/mL). 24 hours later, bFGF (5 ng/mL) was added. On the second day of differentiation, ACTIVIN A, SB-431542 (6 μM), CHIR99021 (3 μM), and/or IWP2 (3 μM) were added. On the third day, EBs were changed to StemPro-34 media supplemented as above, with bFGF (5 ng/mL) and VEGF (15 ng/mL) and treated with either 10 μM of the pan-ALDH inhibitor DEAB (4-Diethylaminobenzaldehyde, Sigma #D86256; “RA-independent”) or 5 μM retinol (ROH, Sigma #R7632; “RA-dependent”). On day 6, IL-6 (10 ng/mL), IGF-1 (25 ng/mL), IL-11 (5 ng/mL), SCF (50 ng/mL), EPO (2 U/mL final) with DEAB or ROH were added. HE was FACS-isolated for terminal assays on day 8 (DEAB) or day 10 (ROH). All differentiation cultures were maintained at 37° C. All embryoid bodies and mesodermal aggregates were cultured in a 5% CO_{2/5}% 02/90% N₂ environment. All recombinant factors are human and were purchased from Biotechne. Analysis of hematopoietic colony potential via Methocult (Stem Cell Technologies) was performed as described previously (Ditadi, A. et al. Nat Cell Biol 17, 580-591, (2015); Kennedy, M. et al. Cell Rep 2, 1722-1735, (2012)).

TABLE 1
Differentiation Scheme

| Reagent | Final Conc. | |
|---|---|---|
| Mesoderm differentiation medium 1-Day 0 | | |
| (On day 0, PS cells were | | |
| cultured on Matrigel coated dishes for | | |
| 24 hours and then resuspended in | | |
| mesoderm differentiation medium 1 | | |
| IMDM + F12 | 75% IMDM | |
| | and 25% F-12 | |
| L-glutamine | 2 | mM |
| Ascorbic acid | 1 | mM |
| monothioglycerol | 4 × 10⁻⁴ M | |
| transferrin | 150 | μg/ml |
| BMP4 | 10 | ng/ml |
| Mesoderm differentiation medium 2-After | | |
| about 24 hours the mesoderm | | |
| differentiation medium 1 is replaced | | |
| with mesoderm differentiation medium 2 | | |
| IMDM + F12 | 75% IMDM | |
| | and 25% F-12 | |
| L-glutamine | 2 | mM |
| Ascorbic acid | 1 | mM |
| monothioglycerol | 4 × 10⁻⁴ M | |
| transferrin | 150 | μg/ml |
| BMP4 | 10 | ng/ml |
| bFGF | 5 | ng/ml |
| Mesoderm differentiation medium 3-After | | |
| about 24 additional hours the | | |
| mesoderm differentiation medium 2 is | | |
| replaced with Mesoderm differentiation | | |
| medium 3 | | |
| IMDM + F12 | 75% IMDM | |
| | and 25% F-12 | |
| L-glutamine | 2 | mM |
| Ascorbic acid | 1 | mM |
| monothioglycerol | 4 × 10⁻⁴ M | |
| transferrin | 150 | μg/ml |
| SB-431542 | 6 | μM |
| CHIR99021 | 3 | μM |
| Hematopoietic specification medium 1-After | | |
| about 24 additional hours the | | |
| mesoderm differentiation medium 3 is | | |
| replaced with Hematopoietic | | |
| specification medium 1 | | |
| Base media | N/A | |
| bFGF | 5 | ng/ml |
| VEGF | 15 | ng/ml |
| Retinol | 5 | μM |
| Hematopoietic specification medium 2-After | | |
| about 3 additional days the | | |
| Hematopoietic specification medium 1 | | |
| is replaced with Hematopoietic | | |
| specification medium 2 | | |
| Base media | N/A | |
| bFGF | 5 | ng/ml |
| VEGF | 15 | ng/ml |
| IL-6 | 10 | ng/ml |
| IGF-1 | 25 | ng/ml |
| IL-11 | 5 | mg/ml |
| SCF | 50 | ng/ml |
| EPO | 2 | U/ml |
| Retinol | 5 | μM |

### Flow Cytometry and Cell Sorting

Cultures were dissociated to single cells, as previously described (Sturgeon, C. M., et al., Nat Biotechnol 32, 554-561, (2014)). All cell sorting was performed in the absence of fetal bovine serum. Cells were washed, labeled, sorted and collected in StemPro-34 media. The antibodies used are all as previously described (Ditadi, A. et al. Nat Cell Biol 17, 580-591, (2015); Sturgeon, C. M., et al., Nat Biotechnol 32, 554-561, (2014); Kennedy, M. et al. Cell Rep 2, 1722-1735, (2012)). KDR (clone 89106), CD4 (clone RPA-T4), CD8 (clone RPA-T8), CD34-APC (clone 8G12), CD34-PE-Cy7 (clone 8G12), CD43 (clone 1G10), CD45 (clone 2D1), CD56 (clone B159), CD73 (clone AD2), CXCR4 (clone 12G5) and CD235a (clone HIR-2). All antibodies were purchased from BD Biosciences (San Diego, CA) except for KDR (Biotechne). Cells were sorted with a FACSAria™ II (BD) cell sorter and analyzed on a LSRFortessa (BD) cytometer.

### Mesoderm Isolation

For isolation of mesodermal populations, day 3 of differentiation WNTd KDR+ CD235a^{neg}CXCR4+/neg and WNTi KDR+ CD235a+ cells were FACS-isolated and reaggregated at 250,000 cells/mL in day 3 media, as above. Cultures were plated in 250 μL volumes in a 24 well low-adherence culture plate, and grown overnight in a 37° C. incubator, with a 5% CO_{2/5}% 02/90% N₂ environment. As specified, RA was manipulated with either 5 μM ROH or ATRA (Sigma #R2625), or 10 μM DEAB. On day 4, an additional 1 mL of RA-supplemented day 3 media was added to reaggregates. On day 6 of differentiation, CD34+ and CD43+ cells from WNTi cultures were FACS-isolated for terminal assays. WNTd cultures were fed as normally, but without additional RA manipulation. CD34+ cells were sorted from all WNTd populations on day 8 of differentiation.

### Endothelial-to-Hematopoietic Transition Assay

CD34+CD43^{neg} hemogenic endothelium was isolated by FACS and allowed to undergo the endothelial-to-hematopoietic transition as described previously (Ditadi, A. et al., Nat Cell Biol 17, 580-591, (2015); Ditadi, A. et al., Methods 101, 65-72, (2016)). Briefly, cells (CD34+CD43^{neg} or CD34+CD43^{neg}CD73^{neg}CXCR4neg cells) were aggregated overnight at a density of 2×10⁵ cells/mL in StemPro-34 media supplemented with L-glutamine (2 mM), ascorbic acid (1 mM), monothioglycerol (MTG, 4×10⁻⁴ M; Sigma-Aldrich), holo-transferrin (150 μg/mL), TPO (30 ng/mL), IL-3 (30 ng/mL), SCF (100 ng/mL), IL-6 (10 ng/mL), IL-11 (5 ng/mL), IGF-1 (25 ng/mL), EPO (2 U/mL), VEGF (5 ng/mL), bFGF (5 ng/mL), BMP4 (10 ng/mL), FLT3L (10 ng/mL), and SHH (20 ng/mL). Aggregates were spotted onto Matrigel-coated plasticware and were cultured for additional 3 or 9 days for WNTi and WNTd cultures, respectively. Cultures were maintained in a 37° C. incubator, in a 5% CO_{2/5}% 02/90% N₂ environment. Hemato-endothelial cultures were subsequently harvested by trypsinization, and assessed for hematopoietic potential by Methocult in a 37° C. incubator, in a 5% CO₂/air environment. The experiments were performed in triplicate and the mean (±standard deviation) of the IC₅₀ values calculated for each data set is reported.

### Op9-DL4 Co-Culture for T-Lineage Differentiation

OP9 cells expressing Delta-like 4 (OP9-DL4) were generated and described previously (La Motte-Mohs, R. N. et al. Blood 105, 1431-1439 (2005); Schmitt, T. M. et al., Nat Immunol 5, 410-417 (2004)). 1-10×10⁴ isolated CD34+CD43^{neg} cells were added to individual wells of a 6-well plate containing OP9-DL4 cells, and cultured with rhFlt-3L (5 ng/mL) and rhlL-7 (5 ng/mL). rhSCF (30 ng/mL) was added for the first 5 days. Cultures were maintained at 37° C., in a 5% CO₂/air environment. Every five days co-cultures were transferred onto fresh OP9-DL4 cells by vigorous pipetting and passaging through a 40 pm cell strainer. Cells were analyzed using a LSRFortessa flow cytometer (BD), as indicated.

### Gene Expression Analyses

Total RNA was prepared for whole-transcriptome sequencing using the Clontech SMARTer kit and was sequenced using an Illumina HiSeq 2500 with 1×50 single reads. Reads were aligned to hg19 using STAR and gene counts were obtained using Subread. TMM normalization and RPKM counts were calculated using EdgeR. Gene Set Enrichment Analysis (GSEA, version 4.0.1) and the Database for Annotation, Visualization, and Integrated discovery (DAVID, version 6.8) were used for differential expression analysis. Morpheus (software.broadinstitute.org/morpheus) was used to create heatmaps and perform hierarchical clustering (one minus the Pearson correlation with average linkage). Bulk RNA-seq comparison to scRNA-seq was performed using the SingleR package (version 1.0.1)(Aran, D. et al., Nat Immunol 20, 163-172, (2019)) implemented in R (version 3.5.1). qRT-PCR was performed as previously described (Sturgeon, C. M., et al., Nat Biotechnol 32, 554-561, (2014)). Briefly, total RNA was isolated with the RNAqueous RNA Isolation Kit (Ambion), followed by reverse transcription using random hexamers and Oligo (dT) with Superscript III Reverse Transcriptase (Invitrogen). Real-time quantitative PCR was performed on a StepOnePlus thermocycle (Applied Biosystems), using Power Green SYBR mix (Invitrogen). Primers used include: ALDH1A2 (5′-TTGCATTCACAGGGTCTACTG-3′ (SEQ ID NO:1) and 5′-GCCTCCAAGTTCCAGAGTTAC-3′)(SEQ ID NO:2) and CYP26A1 (5′-CTGGACATGCAGGCACTAAA-3′ (SEQ ID NO:3) and 5′-TCTGGAGAACATGTGGGTAGA-3′) (SEQ ID NO:4). Gene expression was evaluated as DeltaCt relative to control (ACTB). For globin analysis, the following TaqMan assays (Applied Biosystems) were used: HBB (Hs00747223_g1), HBE1 (Hs00362215_g1), HBG1/2 (Hs00361131_g1), and GAPDH (Hs02786624_g1).

### ScRNA-Seq Analyses

Cells from each day 3 differentiation culture condition were methanol-fixed as previously described (Alles, J. et al., BMC Biol 15, 44, (2017)). Libraries were prepared following the manufacturer's instruction using the 10× Genomics Chromium Single Cell 3′ Library and Gel Bead Kit v2 (PN-120237), Chromium Single Cell 3′ Chip kit v2 (PN-120236), and Chromium i7 Multiplex Kit (PN-120262). 17,000 cells were loaded per lane of the chip, capturing >6000 cells per transcriptome. cDNA libraries were sequenced on an Illumina HiSeq 3000. Sequencing reads were processed using the Cell Ranger software pipeline (version 2.1.0). Using Seurat (version 3.0.2) implemented in R (version 3.5.1), the dataset was filtered by removing genes expressed in fewer than 3 cells, and retain cells with unique gene counts between 200 and 6000. The remaining UMI counts were log-normalized and mitochondrial UMI counts were regressed out. Principal component analysis was used to generate t-distributed stochastic neighbor embedding (t-SNE) and uniform manifold approximation and project (UMAP) plots. Monocle (version 2.10.1) was used for pseudotime analysis. First size factors and dispersions were estimated, and then genes were filtered with expression <0.1 and those not expressed in >10 cells. Doublets were removed by filtering out cells with <4389 and >24813 total RNA. Cell clustering and trajectory construction were performed using an unsupervised approach.

### Data Availability

All gene expression analysis datasets are available in the Gene Expression Omnibus (GEO) under the accession numbers GSE139853 or BioProject #PRJNA352442 and #PRJNA525404 each of which are incorporated herein by reference in their entirety.

### Results

### (i) WNTi and WNTd Cultures are Transcriptionally Distinct.

As Hematopoietic development during embryogenesis is comprised of multiple spatio-temporally regulated hematopoietic programs, each regulated by BMP, WNT, NOTCH, and RA, much of which is recapitulated by hPSC differentiation. By using a stage-specific WNT and ACTIVIN signal differentiation approach, hPSCs can be specified, in a WNT-independent (WNTi) manner, towards a rapidly emerging, NOTCH-independent CD43+ primitive hematopoietic population, as well as a HOXA^{low/neg} CD34+ HE. While WNTi HE is partially NOTCH-dependent and harbors erythroid, myeloid, and granulocytic potential, it lacks T-lymphoid potential, and its resultant BFU-E lack HBG expression, consistent with extra-embryonic hematopoiesis. Conversely, through a WNTd process, hPSCs give rise to NOTCH-dependent HOXA+ HE with definitive erythroid-myeloid-lymphoid potential, consistent with intra-embryonic definitive hematopoiesis. Thus, this stage-specific differentiation platform yields extra-embryonic-like or intra-embryonic-like hematopoiesis in a WNTi or WNTd manner, respectively. However, all these hPSC-derived populations are obtained in an RA-independent manner, as these are chemically-defined conditions, with no exogenous RA. Similarly, manipulation of RA signaling on hPSC-derived HE and its downstream progeny have failed to yield functional improvements. Therefore, the identification of an RA-dependent hematopoietic program has remained elusive.

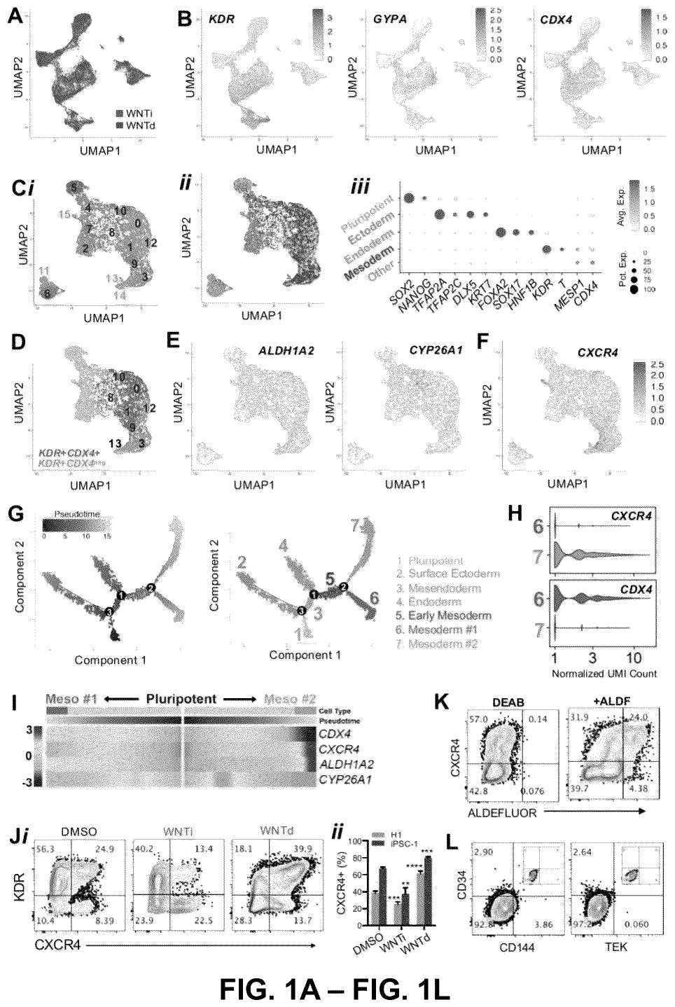

As precise mesodermal patterning is critical for specifying ontogenically-distinct hematopoietic programs, therefore it was first sought to form a better understanding of the mesodermal population(s) obtained during early WNT-mediated differentiation. A single cell (sc)RNA-seq on the day 3 of differentiation cultures under WNTi or WNTd conditions was performed. Following processing with Seurat, WNTi and WNTd cultures exhibited significant overall transcriptional similarity, as evidenced by proximal clustering of the two datasets (FIG. 1A). As expected, a subset of KDR+ cells from the WNTi culture exclusively expressed GYPA (CD235a), identifying it as early extra-embryonic-like hemogenic mesoderm (FIG. 1B). Similarly, CDX4, which regulates the development of hPSC-derived intra-embryonic-like HE, was not expressed in all WNTd KDR+ cells (FIG. 1B), suggesting that definitive hematopoiesis similarly emerges from a subset of mesoderm. Recapitulating their functional differences, each of these populations was transcriptionally distinct (Gene Expression Omnibus (GEO) under the accession numbers GSE139853 or BioProject #PRJNA352442 and #PRJNA525404).

### (ii) ALDH12A2+ CXCR4+ Populations.

To identify a potential RA-dependent mesodermal progenitor in any of these populations, cells expressing ALDH1A2 were searched for. ALDH1A2 governs enzymatic conversion of retinol to all-trans retinoic acid (ATRA) during embryogenesis, and is essential for intra-embryonic HE development. Therefore, WNTd cells were focused on, as the WNTi hemogenic mesoderm was devoid of ALDH1A2expression. Independent clustering of the WNTd cells revealed separation of germ layer-like populations, including multiple KDR+ mesodermal clusters (FIG. 1C), which can be segregated by differential CDX4 expression (FIG. 1D). Surprisingly, while several clusters expressed ALDH1A2, only a small cluster of CDX4 ^{neg} mesodermal cells (FIG. 1E) had significant enrichment in the entire cluster. In contrast, the CDX4+ALDH1A2+ cells spanning clusters 0 and 10 were likely cardiogenic mesoderm, given their co-expression of MESP1, PDGFRA and CXCR4. Therefore, the remaining CDX4+ clusters (1, 8, and 9) to cluster 13 were compared, which revealed strong differential expression of multiple cell surface markers. Of those, the cell surface marker CXCR4 exhibited the strongest enrichment of ALDH1A2+ cells (FIG. 1F).

Complementary pseudotime analyses of these populations revealed a developmental trajectory that recapitulates early embryogenesis, with sequential, distinct germ layer-like populations emerging, including 2 distinct KDR+ mesodermal populations (FIG. 1G). Consistent with the clustering analyses, each KDR+ branch was subset by the exclusive expression of CXCR4 or CDX4 (FIG. 1H). Furthermore, ALDH1A2 was exclusively expressed within this CXCR4+ population, with concomitant CDX4 downregulation (FIG. 1I). Flow cytometry confirmed that, in both hESC and iPSC lines, CXCR4 was differentially expressed within day 3 KDR+ cells, and that expression of CXCR4 was regulated by WNT signaling (FIG. 1H). Critically, Aldefluor analysis confirmed that ALDH expression is enriched at the protein level within this CXCR4+ mesoderm (FIG. 1K). Finally, these populations were immunophenotypically CD34^{neg} CD144^{neg} TEK^{neg} (FIG. 1L), establishing them as a mesodermal population that precedes hemato-endothelial specification. Collectively, these observations reveal that at least two hemogenic mesodermal populations exist following WNTd differentiation conditions, with CXCR4+ cells uniquely expressing ALDH1A2.

(iii) Characterization of WNTi KDR+ CD235a+ cells, and the WNTd KDR+ CXCR4neg and KDR+ CXCR4+ Populations.

To further characterize these populations, whole-transcriptome analyses on day 3 WNTi KDR+ CD235a+ cells, and the WNTd KDR+ CXCR4^{neg} and KDR+ CXCR4+ populations was performed. Hierarchal clustering revealed that WNTi CD235a+ cells were distinct from the WNTd KDR+ populations, consistent with its extra-embryonic-like hematopoietic potential. Both WNTd KDR+ populations expressed HOXA genes, consistent with a role for WNT/GSK3p in regulating CDX/HOXA expression in hPSC-derived mesoderm. Interestingly, the KDR+ CXCR4+ population had lower, but not absent, CDXexpression than KDR+ CXCR4^{neg} cells. Consistent with flow cytometric analyses, all three KDR+ populations were transcriptionally distinct from later-emerging HE, as they lacked expression of canonical hemato-endothelial markers, such as CD34, CDH5, RUNX1, TAL1, and MYB, but instead expressed early mesodermal genes, such as TBXT and MIXL1. Finally, this confirmed a striking difference in the expression of RA-related genes between the mesodermal populations, with CYP26A1 enriched in WNTi CD235a+ and WNTd CXCR4^{neg} mesoderm, while ALDH1A2 was exclusively expressed within KDR+ CXCR4+ mesoderm.

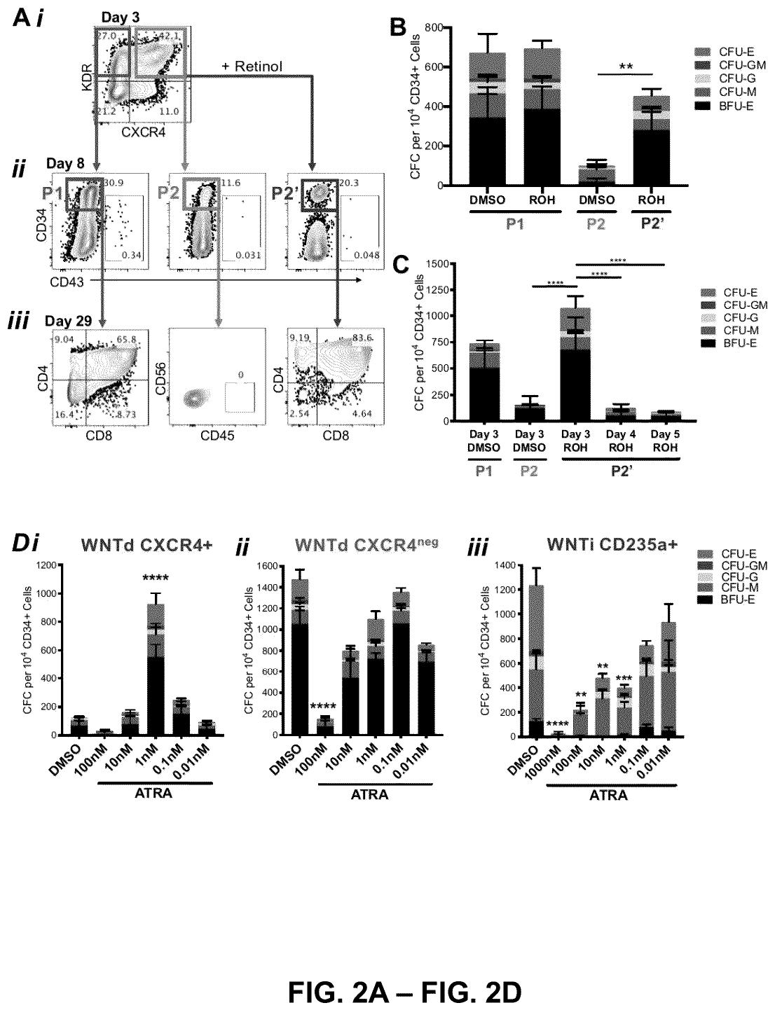

To assess which WNTd KDR+ subset(s) could give rise to HOXA+ definitive HE, each CXCR4₊^{/neg} population was isolated by FACS, and then cultured for an additional 5 days to allow for HE specification (FIG. 2A). Both CXCR4^{ne}g and CXCR4+ populations gave rise to a CD34+CD43^{neg} population (FIG. 2A). However, multilineage definitive hematopoietic potential was exclusively restricted to the CXCR4^{neg} mesoderm, as this exhibited definitive erythro-myeloid and T-lymphoid potential (P1; FIGS. 2A and 2B). In contrast, CD34+ cells derived from the KDR+ CXCR4+ population lacked multilineage hematopoietic potential (P2; FIGS. 2A and 2B). This strongly suggests that WNT-mediated definitive hematopoietic specification from hPSCs originates from a KDR+ CXCR4^{neg} CD34^{neg} CDX4+ mesodermal population. Further, as this population expresses CYP26A1, and gives rise to definitive hematopoietic progenitors in the presence of the pan-ALDH inhibitor DEAB (not shown), this strongly suggests that this is an RA-independent (RAi) hematopoietic progenitor.

Given that the CXCR4+ population exhibited no hematopoietic potential, but was enriched in ALDH1A2 expression, it was hypothesized that this population may exhibit an RA-dependent response. Therefore, freshly isolated KDR+ populations were cultured with retinol (ROH; FIG. 2A). Critically, this treatment resulted in the specification of CD34+ HE that harbored definitive erythroid, myeloid, and lymphoid hematopoietic potential (P2′; FIGS. 2A and 2B). Interestingly, this RA-mediated response was temporally-restricted, as only treatment of freshly isolated CXCR4+ mesoderm on day 3 of differentiation, but not thereafter, resulted in the specification of HE (FIG. 2C). Therefore, a KDR+ CD34^{neg} CXCR4+ mesodermal population harbors stage-specific, RA-dependent (RAd), definitive hematopoietic potential.

ATRA has been identified as a developmentally-relevant signaling regulator, including as a negative regulator of extra-embryonic hematopoiesis. Therefore it was asked whether ATRA would similarly specify functional HE from WNTd CXCR4+ mesoderm. Titration of ATRA on isolated KDR+ CXCR4+ mesoderm revealed 1 nM exhibiting robust specification of definitive HE, but concentrations lower than 1 nM and higher than 10 nM failed to specify HE from this population (FIG. 20), indicating that a narrow range of RA signaling is required to establish an RAd hematopoietic program. However, 1-10 nM ATRA exhibited no significant effect on definitive hematopoietic development from WNTd KDR+ CXCR4^{neg} mesoderm, while >100 nM was repressive to HE specification (FIG. 20). In sharp contrast, >1 nM ATRA was repressive to extra-embryonic-like HE specification from WNTi CD235a+ mesoderm (FIG. 20), consistent with a repressive role of RA signaling on extra-embryonic hematopoiesis.

(iv) Characterization of HE that is Specified from CXCR4+ Mesoderm.

It was next sought to better understand the HE that is specified from CXCR4+ mesoderm. hPSC-derived definitive HE has been described as a NOTCH-dependent CD34+CD43^{neg} CD73^{neg} CXCR4^{neg} population. To similarly characterize the RAd HE, WNTd differentiation cultures were treated with either DEAB or ROH on day 3 of differentiation to obtain either RAi or RAd definitive hematopoiesis, respectively. Each population gave rise to a CD34+CD43^{neg} population, which could be subset by CD73 and CXCR4 expression. Critically, multilineage hematopoietic potential of both RAi and RAd HE was found within a NOTCH-dependent CD34+CD43^{neg} CD73^{neg} CXCR4^{neg} population. Notably, RAd HE gave rise to significantly more erythro-myeloid CFC potential than RAi HE and the resultant BFU-E exhibited higher expression of fetal (HBG) globin than BFU-E derived from RAi definitive HE, suggesting that, while both progenitors give rise to a fetal-like definitive hematopoietic program, the RAd definitive may be functionally distinct.

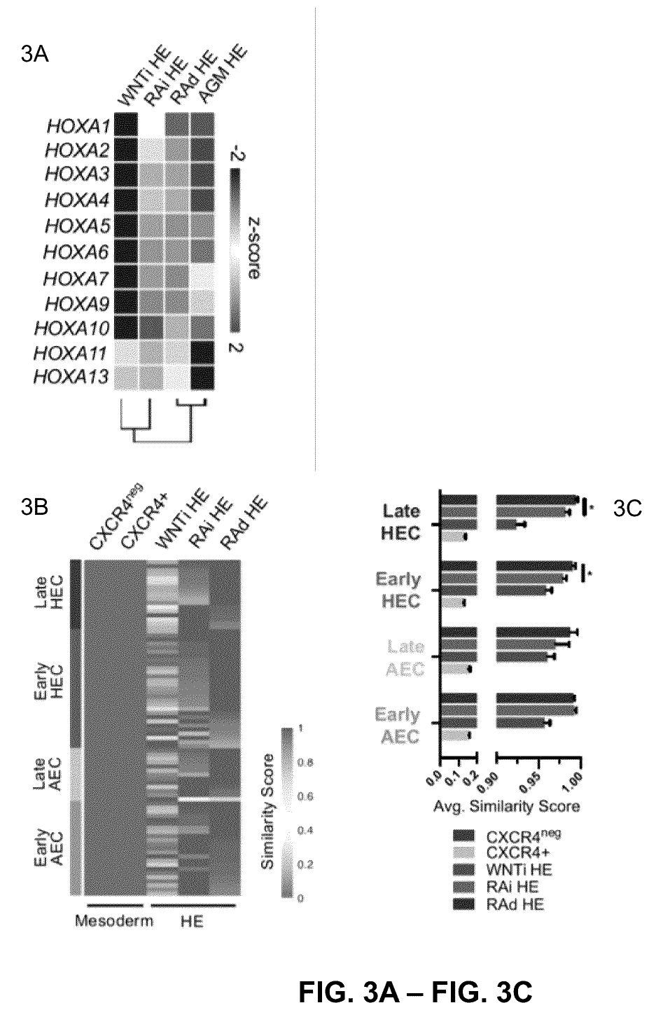

To further asses how these different HE populations compare to each other, whole-transcriptome analyses was performed on each CD34+CD43^{neg} CD73^{neg} CXCR4^{neg} HE. RAi and RAd HE shared a majority of expressed genes and are more similar to each other than to WNTi HE. However, despite this striking similarity, Gene Set Enrichment Analysis (GSEA) revealed that each HE harbored unique transcriptional signatures, with RAd HE being enriched in histone modification and RNA splicing pathways, suggesting complex genetic differences may exist between these populations. To better understand physiological relevance of these differences, hPSC-derived HE was compared to CD34+CD90+CD43^{neg} cells from 5-week human AGM. RAi and RAd HE both expressed hemato-endothelial genes similar to that of primary fetal tissue but had vastly different expression of metabolic genes, which could be reflective of differences between in vitro cultured cells and their primary in vivo correlates. Importantly, however, HOXA expression between each hPSC-derived HE was distinct, with RAd HE exhibiting higher expression of posterior and medial HOXA genes (FIG. 3A), consistent with a more fetal-like expression pattern.

Given the heterogeneity within fetal AGM tissue, which is comprised of both endothelium and HE, it was next utilized the recently-described human fetal HE scRNA-seq dataset, for comparison against hPSC-derived RAi and RAd HE. Included in the analysis was “early” (Carnegie Stages (CS)10/11) and “late” (CS13) intra-embryonic populations of arterial endothelium, and transcriptionally-defined HSC-competent HE cells (FIG. 3B). As expected, fetal HE had no similarity to WNTd mesodermal populations, and relatively low similarity to extra-embryonic-like WNTi HE (FIG. 3B). In sharp contrast, nearly all of the fetal HE cells had a positive correlation when compared to hPSC-derived RAi and RAd HE. However, the RAd HE had the highest similarity to fetal “late” HE (FIG. 3B-3C), suggesting that RAd HE is the most transcriptionally similar to HSC-competent HE, in comparison to any other hPSC-derived HE population. Genes contributing to this high similarity score included many small RNAs, medial HOXA genes, lymphocyte-related genes, and erythro-myeloid-related genes, consistent with these HE populations harboring multi-lineage potential.

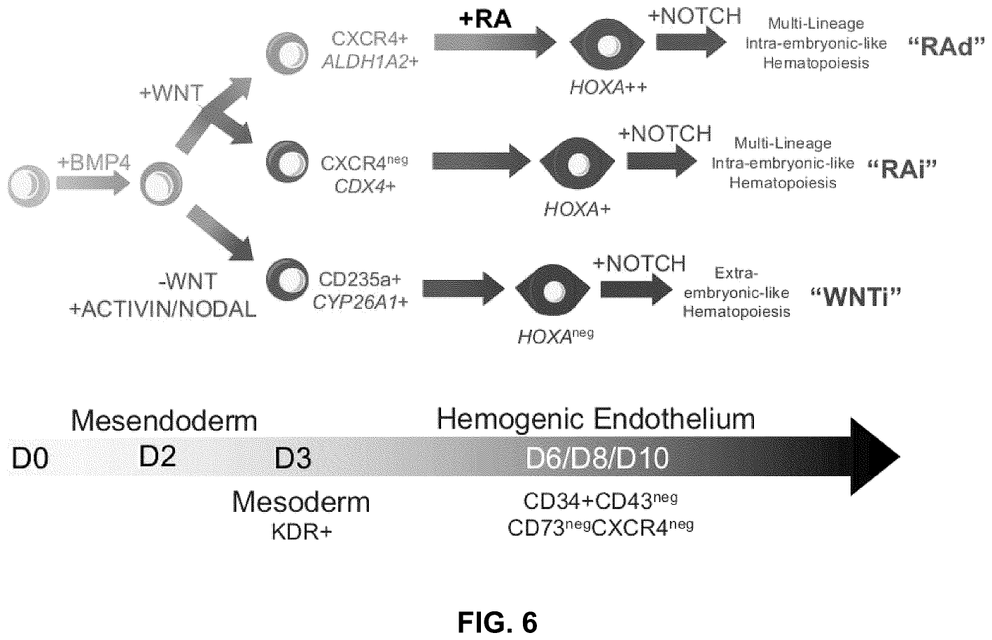

These complementary analyses provide new insight into the multiple, distinct hematopoietic progenitors that can be obtained from hPSCs (FIG. 6). Notably, these studies demonstrate that hPSC-derived hematopoietic potential is restricted to distinct immunophenotypic KDR+ CD34^{neg} mesodermal subpopulations, which are specified very rapidly within differentiation cultures. This is reminiscent of a similar developmental trajectory of cardiomyocyte specification from hPSCs, suggesting that major cell fates are specified immediately following a gastrulation-like stage in differentiation cultures. There are several lines of evidence that suggest that hemogenic specification is a very early event in the murine conceptus, and nascent Gata1+ mesoderm is restricted to be extra-embryonic in hematopoietic potential, similar to hPSC-derived WNTi CD235a+ mesoderm. Each hPSC-derived mesodermal population gives rise to an immunophenotypically similar HE population, but each of which are functionally and transcriptionally distinct. The development of functionally distinct HE is consistent with the identification of HSC-independent HE in the murine yolk sac and human embryo proper.

### CONCLUSION

Previous work demonstrated that NOTCH-dependency is a distinguishing characteristic of WNTd CD34+ HE. Here, it was demonstrate that, while WNTi CD43+ EryP-CFC progenitors are NOTCH-independent, as expected, WNTi HOXA^{low/neg} HE, which harbors erythroid and macrophage/granulocyte potential, is partially NOTCH-dependent. Thus, a requirement for NOTCH cannot be used to distinguish between various hPSC-derived HE populations. However, the lack of HOXA expression in this population identifies it as an extra-embryonic-like progenitor, and its granulocyte potential suggests this WNTi HE may be the equivalent to the murine EMP. Conflicting with this interpretation, however, is the erythroid potential of WNTi HE, as its resultant BFU-E expresses similar levels of HBE to EryP-CFC, while the BFU-E obtained from human yolk sacs at developmental stages consistent with the EMP do not exhibit similar HBE expression. Thus, the in vivo correlate(s) of hPSC-derived WNTi HE remains unclear.

Similar to NOTCH, RA has been identified as a critical regulator of HSC development. However, confounding its use in hPSC differentiation, exogenous RA has been identified as inhibitory to extra-embryonic hematopoiesis. The identification of a mesodermal population that positively responds to a narrow concentration range of ATRA, but is inhibited at higher concentrations indicate that, at physiologically-relevant concentrations found during gastrulation, ATRA may not be inhibitory to extra-embryonic-like hematopoiesis.

Here, it is provided an additional resolution to the hematopoietic potential of WNTd differentiation cultures. It was previously reported that hPSC-derived WNTd HE expresses medial HOXA genes, indicating that this population is intra-embryonic-like. Here it was also observed HOXA expression in HE from similar differentiation conditions, which was identified as RAi definitive hematopoiesis, as it can be obtained in the absence of RA signalling. However, this RAi HE has anterior enrichment of HOXA expression, whereas RAd HE has more posterior and medial HOXA expression, giving it a higher similarity to primary HSC-competent HE. Collectively, these observations have identified a novel ontogeny for multilineage, NOTCH-dependent, RA-dependent definitive HE, and have identified the critical stage-specific nature of its specification from hPSCs. Given its functional and transcriptional similarity to an intra-embryonic population that harbors HSC-competent HE, it is anticipated that this methodology will be of great use to the regenerative medicine community, to better understand the development and regulation of embryonic hematopoiesis, disease modeling studies, and in the pursuit of an hPSC-derived HSC.

### Example 2: Exemplary Method to Develop Retinoic Acid-Dependent Hematopoiesis from Human Pluripotent Stem Cells

The following example describes exemplary methods useful to generate retinoic acid-dependent hematopoietic progenitors from human pluripotent stem cells.

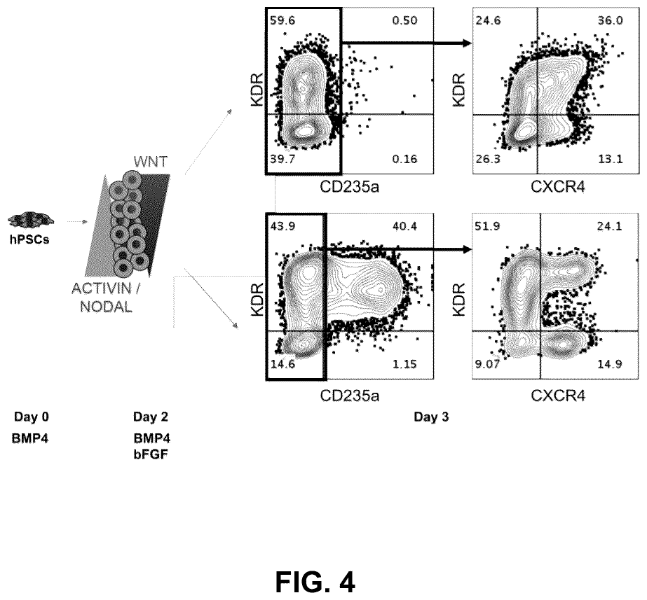

hPSCs, which encompasses both human embryonic stem cells (hESCs) and human induced pluripotent stem cells (hiPSCs), are cultured until 70% confluence. These cells are then removed from these conditions, dissociated into clumps (termed “embryoid bodies”), and then further cultured under hypoxic conditions (e.g., 5% O₂, 5% CO₂). From days 0-3 of differentiation, embryoid bodies are exposed to recombinant human BMP4. On days 1-3, bFGF is added to the differentiation media. On day 2, fresh media is replaced, with additional CHIR99021 (a GSK3b antagonist to stimulate canonical WNT signaling) and SB-431542 (an ALK inhibitor to suppress all ACTIVIN/NODAL signaling within the culture). After these 3 days of culture, a mesodermal population can be identified by its cell surface expression of KDR/VEGFR2, and lack of expression of CD235a (see e.g., FIG. 4).

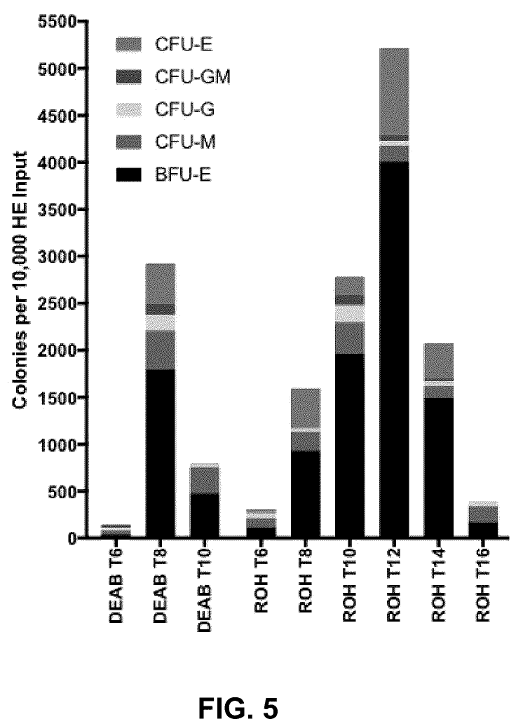

Within this KDR+ CD235a-population, two mesodermal subsets were identified by the expression of CXCR4/CD184 (see e.g., FIG. 4). The emergence of this CXCR4+ population is enhanced by the application of stage-specific WNT signal activation from days 2-3, as above. As described in the example above, gene expression analyses have identified that the CXCR4^{neg} population expresses the gene CYP26A1, suggesting it will not be responsive to RA (see e.g., FIG. 5A). In contrast, the CXCR4+ population expresses the gene ALDH1A2, which suggested that it would convert retinol into RA, and subsequently engage RA-dependent cellular differentiation (see e.g., FIG. 5A). The ALDH1A2 enzyme was expressed and was active, as evidenced by Aldefluor uptake and conversion to a fluorescent compound (see e.g., FIG. 5B).

These cultures are then isolated and further cultured, to give rise to hematopoietic progenitors. Populations are cultured in human serum albumin (HSA) containing media and supplemented with bFGF and VEGF, for an additional 5 days. The resultant cultures result in a CD34+CD43^{neg} CD73^{neg} CD184^{neg} hemogenic endothelial (HE) population that is capable of multi-lineage definitive hematopoiesis, at a clonal level.

When isolated by fluorescence-activated cell sorting (FACS), the day 3 KDR+ CXCR4^{neg} population, upon further culture as above, will similarly give rise to a CD34+CD43^{neg} HE population. This population was capable of multi-lineage definitive hematopoiesis. The addition of a RA inhibitor at any stage of this differentiation process, such as DEAB, has no negative impact resultant definitive hematopoietic specification (not shown). Therefore, definitive hematopoietic progenitors are derived from a KDR+ CXCR4^{neg} mesodermal population, which expresses CYP26A1. Further, this indicates that the definitive hematopoiesis derived from human pluripotent stem cells is retinoic acid-independent.

In contrast, when the day 3 mesodermal KDR+ CXCR4+ population was isolated and cultured in a similar fashion as above, a CD34+ population was obtained on day 8 of differentiation. However, this population completely lacked any hematopoietic potential. Similarly, if the ALDH inhibitor DEAB was added, a CD34+ population was obtained, but completely lacked any hematopoietic potential (not shown). Critically, if the RA precursor, retinol, was added on day 3 of differentiation to these KDR+ CXCR4+ cells, a CD34+ HE population was obtained on day 8 of differentiation. This HE population is capable of erythro-myeloid-lymphoid multilineage hematopoiesis. Therefore, this HE is representative of RA-dependent definitive hematopoiesis, and is derived from a KDR+ CXCR4+ mesodermal cells that express ALDH1A2.

This RA-dependent HE is highly dependent on the correct temporal application of RA signaling. When applied at day 3 of differentiation to isolated KDR+ CXCR4+ mesoderm, RA-dependent HE is specified. However, if RA signaling is applied 1 or 2 days later (day 4 or 5 of differentiation), CD34+ cells are obtained, but these completely lack hematopoietic potential. Therefore, there is a critical stage-specific role for RA signaling in the specification of this HE population.

Obtaining this RA-dependent HE does not require FACS isolation of KDR+ CXCR4+ mesoderm. If RA signaling is applied to bulk differentiation cultures on day 3 of differentiation, which possess a KDR+ CXCR4+ subset, these cells will respond to the RA agonist and specify a CD34+ HE population that persists from days 8-16 of differentiation (see e.g., FIG. 5).

To-date, there have been many published attempts to identify a RA-dependent HE from hPSCs. However, none have elegantly manipulated BMP4, WNT, ACTIVIN/NODAL and RA in the correct temporal order. In contrast, here, a unique, stage-specific method to generate RA-dependent definitive hematopoietic progenitors from hPSCs has been identified. Further, mesodermal population that gives rise to these CD34+ hematopoietic progenitors has also been identified. This is summarized in the schematic (see e.g., FIG. 6).

These studies demonstrate the identification of CXCR4+ mesoderm. It was discovered that definitive hematopoietic KDR+ mesoderm can be subset by CXCR4 expression. All hPSC-derived definitive hematopoiesis characterized to-date originates from a CXCR4negative subset. But the CXCR4+ mesoderm appears poised to respond to retinoic acid signaling. Furthermore, these studies demonstrate CXCR4+ mesoderm responds to retinoic acid signaling, and yields CD34+ definitive hematopoietic progenitors. Multi-lineage definitive hematopoiesis and elevated HOXA gene expression after ROH treatment were shown. It was further discovered that the timing was critical-RA signaling must be received on day 3 of differentiation, no later.

### Example 3: Characterization and Specificity of hPSC-Derived RA-Dependent HE

The following example describes experiments which functionally characterize hPSC-derived RA-dependent HE, to define the specification of hPSC-derived HE populations.

Human Pluripotent Stem Cell (hPSC)-Derived Hematopoietic Stem Cells (HSCs) and their Potential for Regenerative Medicine

HSCs are functionally defined as multipotent stem cells that can provide long-term reconstitution of the entire lymphoid/myeloid hematopoietic system after transplantation into a myeloablated adult recipient. This property has made HSC transplantation a powerful tool in the treatment of various blood disorders. But not all patients are able to receive this life-saving treatment (reviewed in (Clapes T, et al., Regenerative medicine; 7(3):349-68 (2012); Spitzer T R, et al., Cytometry Part B, Clinical cytometry; 82(5):271-9 (2012)). hPSCs (comprised of embryonic stem cells (ESCs) and induced pluripotent stem cells (iPSCs)) differ from HSCs because the fidelity of in vitro gene-correction can be safely assessed before use (Slukvin, II, Blood; 122(25):4035-46 (2013)), and they can be expanded indefinitely in the petri dish, with the potential to differentiate into patient-specific HSCs.

Unfortunately, while there have been multiple studies documenting xenotransplantation of hPSC-derived hematopoietic progenitors, the levels of long-term engraftment observed have been low, and in most cases restricted to the myeloid lineage. Our recently described stage-specific differentiation approach of hPSCs robustly generates CD34+ definitive hematopoietic stem/progenitor cells (HSPCs) with NOTCH-dependent, clonal multi-lineage potential. However, these CD34+ cells similarly lack HSC potential, and require the expression of multiple transgenes for HSC-like function (Sugimura R, et al., Nature; 545(7655):432-8 (2017)). Herein is described how to obtain a better understanding of the temporal signaling requirements of definitive hematopoietic development, so as to recapitulate these processes in vitro, and ultimately obtain transgene-free HSCs from hPSCs. In turn, this is beneficial to multiple scientific communities, enabling the modeling of developmental processes and hematopoietic diseases, and to ultimately develop specific cellular therapies.

Recapitulation and Study of Hematopoietic Development with a Tractable Model System.

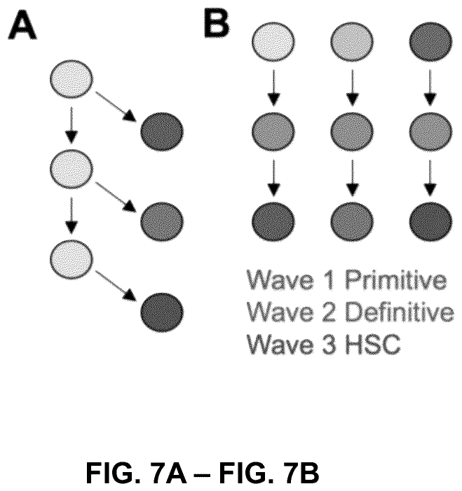

All blood cells originate from an embryonic, developmental intermediate within the vasculature, termed “hemogenic endothelium” (HE). To better understand hematopoiesis, a need exists to understand the development of HE. To-date, there remains debate regarding the mesodermal origin(s) of HE, thought to originate from either a common mesodermal population that yields all HE progenitors (see e.g., FIG. 7A), or, that each wave of hematopoietic development originates from distinct mesodermal subsets (see e.g., FIG. 7B). This problem is difficult to study in gastrulation-stage embryos, with small amounts of tissue, and as more recently demonstrated, limiting numbers of blood progenitors per embryo. Here, using a scalable and tractable model system that recapitulates early development, we present evidence that each hematopoietic program originates from a phenotypically distinct mesodermal population, and that hPSCs allow us to isolate and characterize each of them. With this ability, we can now address complex mechanistic questions that are otherwise difficult to perform in the early embryo.

### Identification and Separation of Developmental Hematopoietic Programs.

Hematopoietic development during embryogenesis is a tightly controlled spatio-temporal process. However, many hPSC differentiation approaches do not temporally introduce signals from the key pathways required for definitive hematopoietic specification, resulting in a mixture of hematopoietic progenitors skewed towards yolk sac-like hematopoiesis. As these are immunophenotypically indistinguishable from their definitive, intra-embryonic-like counterparts, it is difficult to subsequently deconvolute the regulation of definitive HSPC specification. In contrast, our tractable, stage-specific differentiation approach takes into consideration key developmental stages that harbor differential signal requirements, and has identified corresponding cell surface markers of the signal-responsive progenitors of each program. Provide here is evidence for a novel mesodermal progenitor population, that is dependent on RA signaling prior to the specification of HE for the emergence of definitive HSPCs. These studies are the first to identify three different ontogenic origins for HE, that diverge within very early mesoderm, and each can be distinguished by the differential expression of CD235a and CXCR4. As such, an unprecedented degree of resolution is now available to study the ontogeny of HE, a rare but important developmental intermediate, from its earliest identifiable progenitors. Collectively, this approach will generate a “developmental road map” for in vitro hematopoiesis, that can be directly translated into the study of development and disease, and is easily accessible to all research laboratories.

### Identification of Early Mesoderm as Critical to Hematopoietic Specification.

HSC specification from HE requires RA signaling. However, most hPSC-derived HE differentiation strategies do not employ RA signal manipulation, or, they apply RA signaling to heterogeneous populations of equivalently-staged HE/HSPCs, making it difficult to understand the role of RA in hPSC-derived hematopoiesis. To faithfully recapitulate HE development in vitro, essential signal combinations, such as WNT and RA must be present, not only in the correct temporal order, but must also be applied to the appropriate mesodermal progenitor. For example, Lee et al., recently demonstrated a temporally-specific requirement for RA signaling within hPSC-derived subsets of mesoderm, resulting in dramatically different cardiomyocyte subtype generation. It has been recently demonstrated that neural “primary regionalization” is established earlier than previously thought, during gastrulation-like stages of ESC differentiation. Thus, many critical lineage specification events occur during germ layer specification. Here, it is provided, for the first time, evidence of an unappreciated temporal dependence for RA in the specification of HE with definitive hematopoietic potential, and that this signaling is required within early mesoderm. If RA is not applied to these cells, at the appropriate stage, no definitive hematopoietic progenitors are obtained. These studies can provide sorely needed critical insight into the temporal regulation of definitive hematopoietic development.

### New Resolution Added to Old Pathways.

The contribution of Cdx and Hox genes to embryonic hematopoiesis has been well-documented. This has led to the development of a hPSC stage-specific differentiation method to obtain WNT-dependent HE that expresses, albeit at low levels, the same HOXA genes that are found in the intra-embryonic vasculature that harbors HSC-competent HE.

Previous studies used the complementary systems of PSCs and murine embryos to study primitive hematopoietic development, and our research is at the forefront in the development of hPSC directed differentiation approaches, having identified the unique roles for ACTIVIN, WNT and NOTCH in hPSC-derived hematopoiesis. Further, we have recently demonstrated the utility of these approaches in both understanding developmental processes, and in modeling early-onset disease.

As described here, we can improve the efficiency of specifying physiologically relevant definitive hematopoietic progenitors from hPSCs, which may in turn be used for precision hematologic therapies, and modeling disease. As such, this work, intersecting developmental hematopoiesis, hPSC differentiation, and genomic expression analyses, can make a significant impact in the field.

### Development of the Hematopoietic System.

From the perspective of the well-characterized murine embryo, hematopoietic development is comprised of at least three spatiotemporally distinct “waves”. The first wave emerges between E7.25-E8.5 in the yolk sac, and is restricted to primitive erythroid, megakaryocyte, and macrophage progenitors, with no HSC potential. The second wave is surprisingly complex. It is comprised of definitive erythroid/myeloid lineages in the yolk sac between E8.25-E11.0, as well as lymphoid potential in the early embryo.

However, this wave does not generate HSCs. Instead, the third wave gives rise to HSCs, in an Aldh1a2-dependent process. While HSCs and “pre-HSCs” are found at multiple locations in the embryo, the best characterized location for HSC specification is the aorta-gonad-mesonephros (AGM) region at E10.5.

### Endothelial Origin for HSPCs.

Lineage tracing studies have shown that all hematopoietic cells originate from an endothelial-like cell, called hemogenic endothelium. The best-characterized source of HE is the ventral wall of the dorsal aorta in the AGM region, wherein nascent HSCs are first detected. HE expresses both endothelium markers and hematopoietic genes, but this co-expression does not necessarily distinguish HE from vascular endothelium. Nascent HSCs arise from HE in a process called the endothelial-to-hematopoietic transition (EHT). This EHT is Notch-dependent wherein cells acquire the expression of the pan-hematopoietic marker CD45, while gradually losing endothelial marker expression. However, not all of these cells are HSCs, but rather a mixture of both HSCs and committed hematopoietic progenitors. The specification and function of this HSC-competent HE is dependent on exposure to RA signaling. Therefore, the identification of an hPSC-derived NOTCH- and RA-dependent HE population is essential for the in vitro generation of HSCs.

### Hematopoiesis in the Human Embryo.

Least understood is primitive hematopoiesis, occurring between 16-19 days post-coitum. This is followed at 28-35 dpc by the emergence of HSC-independent granulocyte-monocyte and HBG+ erythroid progenitors in the yolk sac. Within the AGM, HE undergoing the EHT is visible in the dorsal aorta between 27-42 dpc, where the first detectable HSCs are found between 32-33 dpc.

These parallels across species support that there are at least 3 distinct waves of human hematopoietic development, which we can now recapitulate in vitro with hPSCs (as described herein).

### Hpsc Differentiation System to Model Hematopoietic Development.

We have developed an in vitro system to recapitulate the earliest stages of hematopoietic development. In the embryo, mesodermal cells execute at least three major identity changes as they develop into hematopoietic progenitors, and our system captures all of them via stage-specific signal manipulation. Briefly, in Stage 1, mesoderm is patterned with WNT signal small molecule agonists (CHIR99021) or antagonists (IWP2), to specify either WNT-dependent (WNTd) definitive, or WNT-independent (WNTi) primitive hematopoietic mesoderm, respectively, and these can be distinguished by CD235a expression. In Stage 2, these mesodermal populations are specified towards CD34+ HE, via VEGF and supporting hematopoietic cytokines. In Stage 3, these cultures can be assessed for their ability to give rise to primitive hematopoietic progenitors, which can be identified by nucleated erythroblasts (EryP-CFC) that express embryonic forms of hemoglobin (HBE1 in the human). Or, CD34+ HE can be assessed for definitive hematopoietic potential, as evidenced by its ability to generate HBG+ erythroblasts, myeloid cells, and T-lymphocytes in a NOTCH-dependent manner. The exclusive separation of these programs in Stage 1 establishes the basis for the hPSC model of hematopoietic specification.

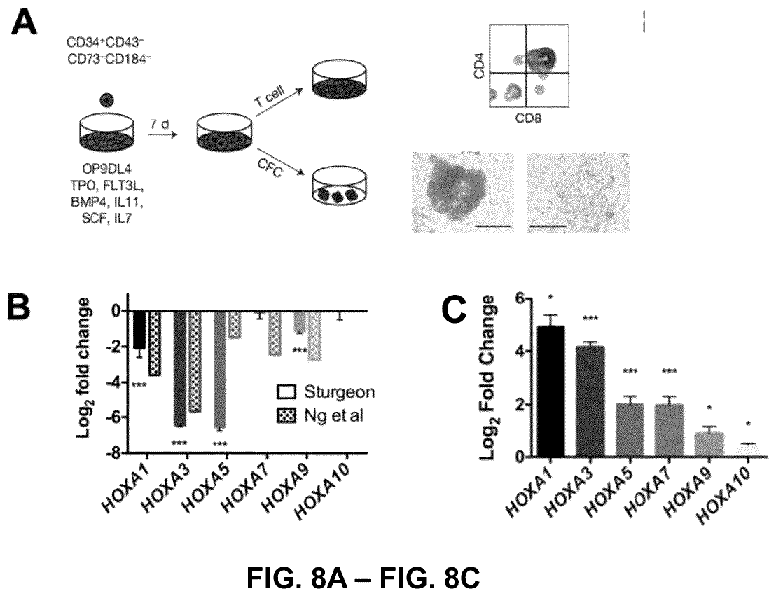

While this WNT-dependent population lacks HSC-like engraftment potential in a xenograft model, through the use of a clonal multi-lineage assay that we developed (see e.g., FIG. 8A), we demonstrated that 10% of this HE possesses bona fide erythro-myelo-lymphoid multi-lineage potential. We then employed whole-transcriptome analyses and genetic engineering to demonstrate that CDX4 is a critical regulator of WNT-mediated definitive hematopoietic specification, consistent with other model systems. Finally, others have found that these WNT-dependent CD34+ cells share significant transcriptional similarity with those found in vivo. However, we have found that this hPSC-derived HE has significantly reduced medial HOXA expression in comparison to its in vivo counterpart (see e.g., FIG. 8B). Despite RA being a critical regulator of medial HOXA expression, application of RA to hPSC-derived HE and HSPCs failed to yield an engraftable HSC population. Thus, the identification of an RA-dependent hPSC-derived HE remained elusive.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, a “population” of cells refers to a group of at least 2 cells, e.g. 2 cells, 3 cells, 4 cells, 10 cells, 100 cells, 1000 cells, 10,000 cells, 100,000 cells or any value in between, or more cells. Optionally, a population of cells can be cells which have a common origin, e.g. they can be descended from the same parental cell, they can be clonal, they can be isolated from or descended from cells isolated from the same tissue, or they can be isolated from or descended from cells isolated from the same tissue sample. Preferably, the population of hematopoietic progenitor cells is substantially purified. As used herein, the term “substantially purified” means a population of cells substantially homogeneous for a particular marker or combination of markers. By substantially homogeneous is meant at least 80%, at least 85%, at least 90%, at least 95%, at least 96%, at least 97%, at least 98%, at least 99% or more homogeneous for a particular marker or combination of markers.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

It should also be understood that, unless clearly indicated to the contrary, in any methods claimed herein that include more than one step or act, the order of the steps or acts of the method is not necessarily limited to the order in which the steps or acts of the method are recited.

1. A method of generating a population of hematopoietic progenitor cells from a bulk differentiation culture, the method comprising:
(i) culturing a population of induced pluripotent stem cells (iPSCs) in a mesoderm differentiation medium comprising a base media supplemented with:
(a) L-glutamine;
(b) ascorbic acid;
(c) monothioglycerol;
(d) transferrin; and
(e) (1) a bone morphogenic protein (BMP), or (2) an activin receptor-like kinase inhibitor and a GSKβ inhibitor,
wherein the mesoderm differentiation medium further comprises supplementation with an activator of WNT signaling or a GSKP inhibitor between day 2 and day 3 of differentiation; and
(ii) culturing a population of cells obtained from step (i) in a hematopoietic specification medium comprising a base media supplemented with:
(a) a FGF,
(b) a VEGF, and
(c) a retinoic acid signaling agent,
from day 3 of differentiation to produce a population of CD34⁺CD43^{neg}CD73^{neg}CD184^{neg} hematopoietic progenitor cells.

2. The method of claim 1, wherein the mesoderm differentiation medium further comprises FGF when the mesoderm differentiation medium comprises a bone morphogenic protein (BMP).

3. The method of claim 1, wherein the BMP is BMP4.

4-6. (canceled)

7. The method of claim 3, wherein the FGF is bFGF.

8. (canceled)

9. The method of claim 1, wherein the mesoderm differentiation medium comprises a GSKβ inhibitor, wherein the GSKβ inhibitor is CHIR99021.

10-12. (canceled)

13. The method of claim 1, wherein the retinoic acid signaling agent is selected from retinol (ROH), a retinoic acid, a retinoic acid receptor (RAR) agonist, a RAR alpha (RARA) agonist, a RAR beta (RARB) agonist, or a RAR gamma (RARG) agonist.

14. The method of claim 1, wherein the cells obtained from (i) are cultured in the hematopoietic specification medium for about 3 days.

15-18. (canceled)

19. A population of CD34⁺CD43^{neg}CD73^{neg}CD184^{neg} hematopoietic progenitor cells, which is produced by the method of claim 1, wherein the population has hematopoietic potential.

20-37. (canceled)

38. The method of claim 2, wherein the BMP is BMP4.

39. The method of claim 13, wherein
(i) the retinoic acid is an all-trans-retinoic acid (ATRA);
(ii) the RARA agonist is AM580;
(iii) the RARB agonist is BMS453; or
(iv) the RARG agonist is CD1530.

40. A population of CD34⁺CD43^{neg}CD^{neg}CD184^{neg} hematopoietic progenitor cells, which is produced by the method of claim 2, wherein the population has hematopoietic potential.

41. A population of CD34+CD43^{neg}CD^{neg}CD184^{neg} hematopoietic progenitor cells, which is produced by the method of claim 3, wherein the population has hematopoietic potential.

42. A population of CD34⁺CD43^{neg}CD^{neg}CD184^{neg} hematopoietic progenitor cells, which is produced by the method of claim 7, wherein the population has hematopoietic potential.

44. A population of CD34⁺CD43^{neg}CD^{neg}CD184^{neg} hematopoietic progenitor cells, which is produced by the method of claim 9, wherein the population has hematopoietic potential.

45. A population of CD34⁺CD43^{neg}CD^{neg}CD184^{neg} hematopoietic progenitor cells, which is produced by the method of claim 13, wherein the population has hematopoietic potential.

46. A population of CD34⁺CD43^{neg}CD^{neg}CD184^{neg} hematopoietic progenitor cells, which is produced by the method of claim 14, wherein the population has hematopoietic potential.

47. A population of CD34⁺CD43^{neg}CD^{neg}CD184^{neg} hematopoietic progenitor cells, which is produced by the method of claim 19, wherein the population has hematopoietic potential.

48. A population of CD34⁺CD43^{neg}CD^{neg}CD184^{neg} hematopoietic progenitor cells, which is produced by the method of claim 38, wherein the population has hematopoietic potential.

49. A population of CD34⁺CD43^{neg}CD^{neg}CD184^{neg} hematopoietic progenitor cells, which is produced by the method of claim 39, wherein the population has hematopoietic potential.


SEQUENCE LISTING (4 sequences)

SEQ ID NO: 1 - DNA, 21 bp, synthetic construct
  misc_feature 1..21: note = SYNTHESIZED
  source 1..21: mol_type = other DNA
ttgcattcacagggtctactg

SEQ ID NO: 2 - DNA, 21 bp, synthetic construct
  misc_feature 1..21: note = SYNTHESIZED
  source 1..21: mol_type = other DNA
gcctccaagttccagagttac

SEQ ID NO: 3 - DNA, 20 bp, synthetic construct
  misc_feature 1..20: note = SYNTHESIZED
  source 1..20: mol_type = other DNA
ctggacatgcaggcactaaa

SEQ ID NO: 4 - DNA, 21 bp, synthetic construct
  misc_feature 1..21: note = SYNTHESIZED
  source 1..21: mol_type = other DNA
tctggagaacatgtgggtaga